(19) United States
(12) Patent Application Publication
(10) Pub. No.: US 2007/0054289 A1
(43) Pub. Date: Mar. 8, 2007

(54) Identification of genes involved in fertility, ovarian function and/or fetal/newborn viability

(75) Inventors: Jose Cibelli, East Lansing, MI (US); Javier Crosby, Providencia (CL); Emilio Fernandez, (US); Arif Kocabas, East Lansing, MI (US); Guilherme Jordao De Magalhaes Rosa, East Lansing, MI (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Michigan State University, East Lansing, MI (US)

(21) Appl. No.: 11/437,797

(22) Filed: May 22, 2006

Related U.S. Application Data
(63) Continuation-in-part of application No. 11/091,883, filed on Mar. 29, 2005.
(60) Provisional application No. 60/556,875, filed on Mar. 29, 2004.

(51) Int. Cl. C12Q 1/00; C12Q 1/68

(52) U.S. Cl. 435/6; 435/4; 800/24

(57) ABSTRACT

A genetic means of determining whether a female subject produces “pregnancy competent” oocytes is provided. The means comprises detecting the level of expression of one or more genes that are expressed at characteristic levels (upregulated or downregulated) in cumulus cells derived from pregnancy competent oocytes. This characteristic gene expression level, or pattern referred to herein as the “pregnancy signature”, also can be used to identify subjects with underlying conditions that impair or prevent the development of a viable pregnancy, e.g., pre-menopausal condition, other hormonal dysfunction, ovarian dysfunction, ovarian cyst, cancer or other cell proliferation disorder, autoimmune disease and the like. Microarrays containing “pregnancy signature” genes or corresponding polypeptides provide another preferred aspect of the invention. Still further, the subject invention can be used to derive animal models, e.g., non-human primate animal models, for the evaluation of the efficacy of putative female fertility treatments. Additionally, an improved RNA amplification protocol is provided herein referred to as the CRL amplification protocol which is suitable for reproducibly amplifying all the RNAs expressed by a cell sample, even when only a few cells are available.

### CROSS REFERENCE TO RELATED APPLICATIONS

This application is a continuation-in-part of U.S. Ser. No. 11/091,883 filed on Mar. 29, 2005. This application further claims the benefit of provisional application No. 60/556,875 filed Mar. 29, 2004. Both of these applications are incorporated herein by reference in their entirety.

### FIELD OF THE INVENTION

The present invention provides genetic methods that provide for the identification of “pregnancy competent” oocytes, i.e., oocytes that when fertilized and transferred to a suitable uterine environment are capable of yielding a viable pregnancy. The present invention further provides genetic methods of identifying subjects, preferably human females having impaired fertility function, e.g., as a result of impaired ovarian function, e.g., as a result of age (menopause) or an underlying disease condition.

Also, the invention provides methods of evaluating the efficacy of a putative fertility treatment based on its effect on the expression of specific genes.

Further, the invention identifies genes which are differentially expressed by cumulus cells that correlate to pregnancy potential of oocytes that are associated therewith.

Further, the present invention provides an improved mRNA amplification protocol that is especially suited for gene expression profiling of biological samples of small quantity, such as cumulus or stem cells.

### BACKGROUND OF THE INVENTION

Currently, there is no available genetic procedures for identifying whether a female subject produces oocytes that are “pregnancy competent”, i.e., which when fertilized by natural or artificial means are capable of giving rise to embryos that in turn are capable of yielding viable offspring when transferred to an appropriate uterine environment. Rather, conventional fertility assessment methods assess fertility e.g., based on hormonal levels, visual inspection of numbers and quality of oocytes, surgical or non-invasive (MRI) inspection of the female reproduction system organs, and the like. Often, when a woman has a problem in producing a viable pregnancy after a prolonged duration, e.g., more than a year, the diagnosis may be an “unexplained” fertility problem and the woman advised to simply keep trying or to seek other options, e.g., adoption or surrogacy. Therefore, providing alternative and more predictive methods for identifying women with fertility problems would be highly desirable. Likewise, novel and improved methods for treating fertility problems would be highly desirable.

Still further, the identification of women with fertility problems, preferably earlier on than by current methods is desirable, as fertility problems may correlate to other health issues that preclude pregnancy, e.g., cancer, menopausal condition, hormonal dysfunction, ovarian cyst, or other underlying disease or health related problems.

### BRIEF DESCRIPTION AND OBJECTS OF THE INVENTION

It is an object of the invention to provide a novel and improved method of detecting infertility problems and the genetic basis thereof.

It is a more specific object of the invention to provide a novel method of detecting female fertility or infertility which method comprises evaluating the capability of oocytes produced by said female to potentially give rise to a viable pregnancy upon fertilization and transferal into a suitable uterine environment, wherein said method involves detecting the levels of expression of specific (“pregnancy signature”) genes or polypeptides encoded thereby.

It is another specific object of the invention to provide a method of evaluating whether a subject produces oocytes capable of giving rise to a viable pregnancy comprising:

(i) measuring the expression levels of genes in a oocyto-derived cell, e.g., a cumulus cell, wherein said genes are expressed or not expressed at characteristic levels (“pregnancy signature”) in cells associated with oocytes capable of yielding a viable pregnancy; and

(ii) detecting the “pregnancy potential” of said oocytes based on the level of similarity of said gene expression pattern to said “pregnancy signature”.

It is another specific object of the invention to identify a female subject putatively having a condition that inhibits or prevents pregnancy by detecting whether said subject produces oocytes associated with cells, e.g., cumulus cells, which do not express one or more genes in a manner characteristic of “pregnancy competent” oocytes; wherein said method comprises detecting the expression of said one or more “pregnancy signature” genes in at least one cell derived from an oocyte isolated from said female subject; and thereby identifying the subject as potentially having a health problem which prevents or precludes fertility based on an abnormal expression pattern of at least one of said “pregnancy signature” genes.

It is another object of the invention to provide a method of evaluating the efficacy of a female fertility treatment which comprises:

(i) treating a female subject putatively having a problem that prevents or inhibits her from having a “viable pregnancy” and

(ii) isolating at least one oocyte from said female subject after said fertility treatment;

(iii) isolating at least one cell from said isolated oocyte, preferably cumulus cell, and detecting the level of expression of at least one gene that is expressed at a characteristic level of expression in “pregnancy competent” oocytes; and

(iv) determining the putative efficacy of said fertility treatment based on whether said gene is expressed at a level characteristic of “pregnancy competent” oocytes as a result of treatment.

It is another object of the invention to provide animal models for evaluating the efficacy of putative fertility treatments comprising identifying genes which are expressed at characteristic levels in cumulus cells derived from pregnancy competent oocytes of a non-human animal, e.g., a non-human primate; and assessing the efficacy of a putative fertility treatment in said non-human animal based on its effect on said gene expression levels, i.e., whether said treatment results in said gene expression levels better mimicking gene expression levels observed in cumulus cells associated with pregnancy competent oocytes, (“pregnancy signature”).

It is another object of the invention to identify specific human genes that are differentially expressed by cumulus cells and other oocyte-associated cells and to assay the expression of one or more of such specific genes by cumulus or other oocyte-associated cells as an indicator of fertility and ovarian function.

It is another object of the invention to provide a novel mRNA amplification protocol especially suited for biological samples of small quantity that combines the use of specific primers, i.e., SMART II oligonucleotide (Clontech, CA) and T7-oloigo(dT)24V promoter primers (Ambion, TX).

### BRIEF DESCRIPTION OF THE FIGURES

FIGS. 1A-1C and 1D-H depict schematically a genetic fertility testing method according to the invention. FIG. 1A shows a freshly ovulated egg containing a polar body, zona pellucida and cumulus cells. FIG. 1B shows the fertilization and transferal of this egg into a uterine environment. FIG. 1C shows the recovery of cumulus cells from the oocytes shown in 1A which are to be used for genetic testing. FIG. 1D-H show the isolation of RNAs from said cumulus cells, microarray analysis of said RNAs, validation of 100 genes by real time RT-PCR, correlation of the levels of expression of said genes (upregulated or downregulated) to the ability of an oocyte to give rise to a viable pregnancy, and the use of this gene expression profile to identify a set of genes, the expression of which correlates to the capability of an oocyte to yield a viable pregnancy (“pregnancy signature”)

FIG. 2 contains a flow chart of the CRL amplification protocol used in the present invention.

FIG. 3 contains a digital RNA gel-like image of total RNA sample isolated from 8 mature oocytes run 3 times.

FIG. 4 contains RT-PCR verification of GeneChip array results for samples as described infra.

FIG. 5 contains selected overrepresented GO biological processes in oocytes identified by EASE.

FIG. 6 contains Venn diagrams depicting intersection of differentially expressed genes in human and mouse oocytes and ESCs.

FIG. 7 contains primers and sequences used to validate RT-PCR microarray experimental results.

FIG. 8 contains selected representations of genes common to human and mouse oocytes with homologs functionally characterized in the mouse oocye (16 genes out of top 100 genes).

FIG. 9 contains 66 unique genes in common between human oocytes, mouse oocytes, hESCs, and mESCs.

FIG. 10 contains genes differentially expressed in human oocytes corresponding to the TGF-beta signaling pathway.

FIG. 11 contains gene that are differentially expressed in human and mouse oocytes that are in the estrogen receptor signaling pathway.

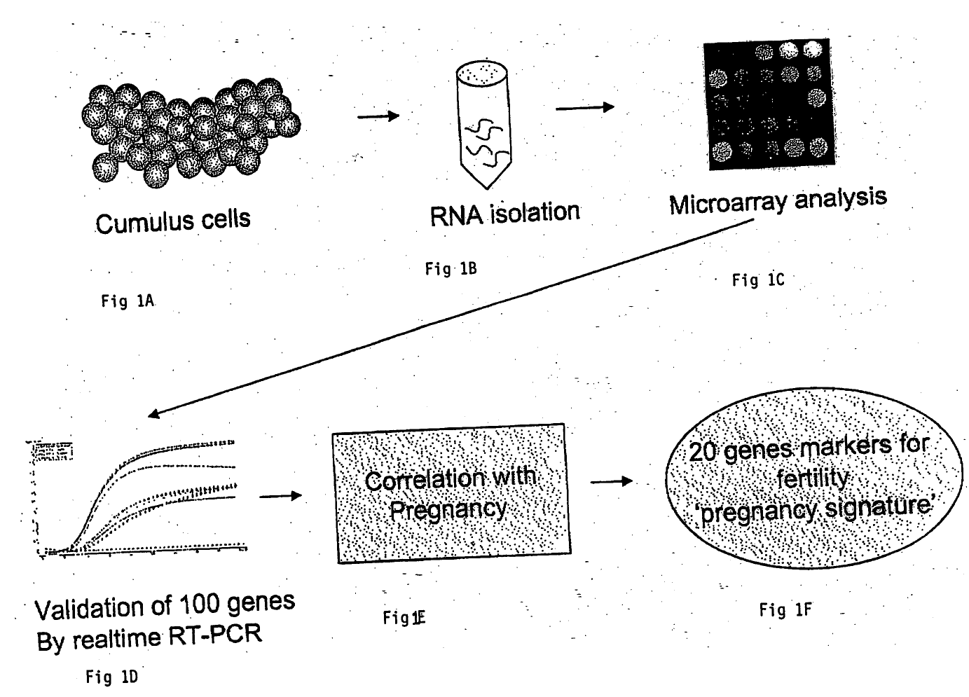

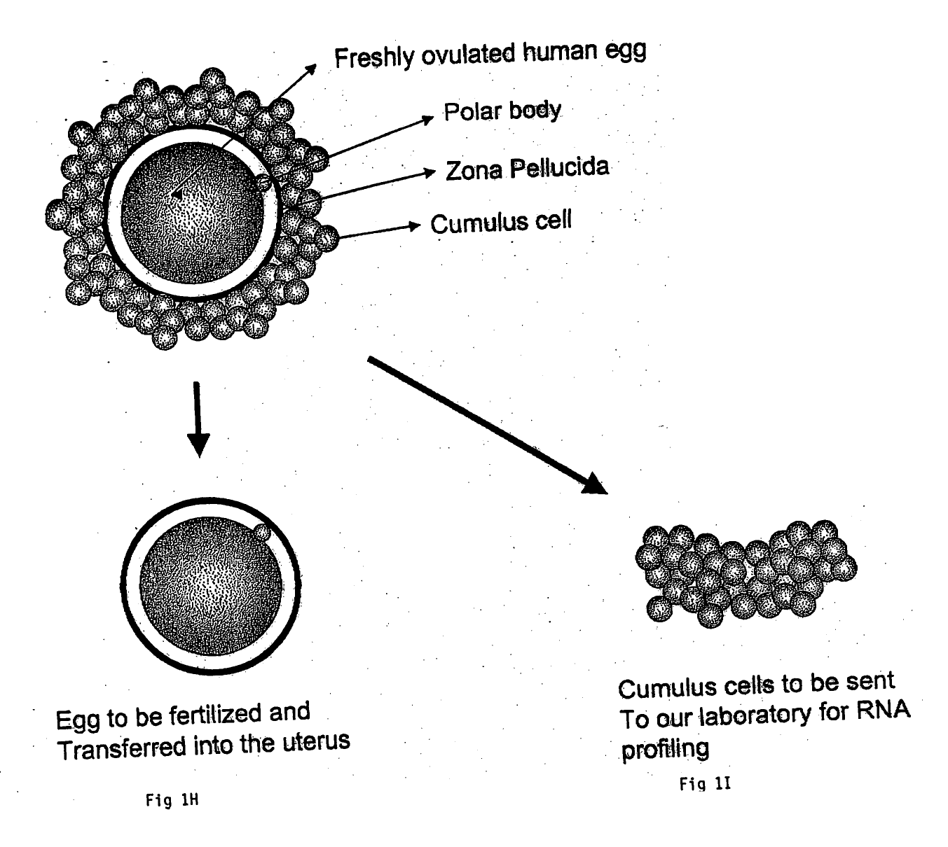

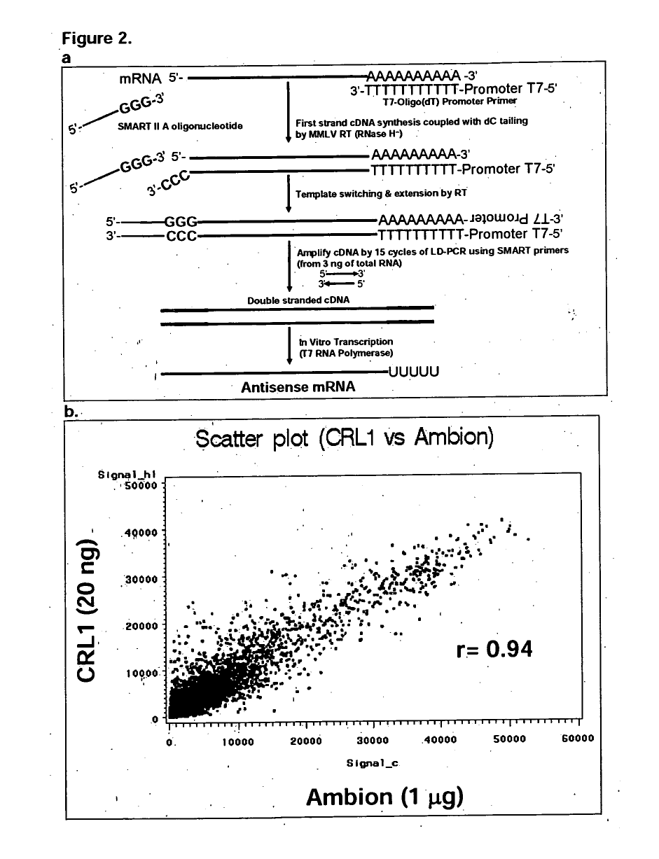

### DETAILED DESCRIPTION OF THE INVENTION

Prior to discussing the invention in more detail, the following definitions are provided. Otherwise all words and phrases in this application are to be construed by their ordinary meaning, as they would be interpreted by an ordinary skilled artisan within the context of the invention.

“Pregnancy-competent oocytes”: refers to a female gamete or egg that when fertilized by natural or artificial means is capable of yielding a viable pregnancy when it is comprised in a suitable uterine environment.

“Viable-pregnancy”: refers to the development of a fertilized oocyte when contained in a suitable uterine environment and its development into a viable fetus, which in turn develops into a viable offspring absent a procedure or event that terminates said pregnancy.

“Cumulus cell” refers to a cell comprised in a mass of cells that surrounds an oocyte. These cells are believed to be involved in providing an oocyte nutritional and or other requirements that are necessary to yield an oocyte which upon fertilization is “pregnancy competent”.

“Differential gene expression” refer to genes the expression of which varies within a tissue of interest; herein preferably a cell from an oocyte, e.g., a cumulus cell.

“Real Time RT-PCR”: refers to a method or device used therein that allows for the simultaneous amplification and quantification of specific RNA transcripts in a sample.

“Microarray analysis”: refers to the quantification of the expression levels of specific genes in a particular sample, e.g., tissue or cell sample.

“Pregnancy signature”: refers to a phrase coined by the inventors which refers to the characteristics levels of expression of a set of one or more genes, preferably at least 5, more preferably at least 10 to 20 genes, and still more preferably, at least 50 to 100 genes, that are expressed at characteristic levels in oocyte cells, preferably cumulus cells, that surround “pregnancy competent” oocytes. This is intended to encompass the level at which the gene is expressed and the distribution of gene expression within cells analyzed.

“Pregnancy signature gene”: refers to a gene which is expressed at characteristic levels by a cell, e.g., cumulus cell, on a “pregnancy competent” oocyte.

“IVF”: refers to in vitro fertilization.

“Zona pellucida” refers to the outermost region of an oocyte.

“Method for detecting differential expressed genes” encompasses any known method for evaluating differential gene expression. Examples include indexing differential display reverse transcription polymorase chain reaction (DDRT-PCR; Mahadeva et al, 1998, J. Mol. Biol. 284:1391-1318; WO 94/01582; subtractive mRNA hybridization (See Advanced Mol. Biol.; R. M. Twyman (1999) Bios Scientific Publishers, Oxford, p. 334, the use of nucleic acid arrays or microarrays (see Nature Genetics, 1999, vol. 21, Suppl. 1061) and the serial analysis of gene expression. (SAGE) See e.g., Valculesev et al, Science (1995) 270:484-487) and real time PCR (RT-PCR). For example, differential levels of a transcribed gene in an oocyte cell can be detected by use of Northern blotting, and/or RT-PCR.

CRL amplification protocol refers to the novel total RNA amplification protocol depicted schematically in FIG. 2 that combines template-switching PCR and T7 based amplification methods. As noted above, this protocol is well suited for samples wherein only a few cells or limited total RNA is available.

Preferably, the “pregnancy signature” genes will be detected by hybridization of RNA or DNA to DNA chips, e.g., filter arrays comprising cDNA sequences or glass chips containing cDNA or in situ synthesized oligonucleotide sequences. Filtered arrays are typically better for high and medium abundance genes DNA chips can detect low abundance genes. In the exemplary embodiment the sample may be probed with Affymetrix GeneChips comprising genes from the human genome or a subset thereof.

Alternatively, polypeptide arrays comprising the polypeptides encoded by pregnancy signature genes or antibodies that bind thereto may be produced and used for detection and diagnosis.

“EASE” is a gene ontology protocol that from a list of genes forms subgroups based on functional categories assigned to each gene based on the probability of seeing the number of subgroup genes within a category given the frequency of genes from that category appearing on the microarray.

As noted above, the present invention preferably provides a novel method of detecting whether a female subject, human or non-human, produces “pregnancy competent” oocytes. The method involves detecting the levels of expression of one or more genes that are expressed or not expressed at characteristic levels by cumulus cells associated with (surrounding) oocytes that are “pregnancy competent”, i.e., which when fertilized by natural or artificial means (IVF), and transferred into a suitable uterine environment are capable of yielding a viable pregnancy, i.e., embryo that develops into a viable fetus and eventually an offspring unless the pregnancy is terminated by some event or procedure, e.g., a surgical or hormonal intervention.

The invention further provides a novel and improved means for amplifying the total RNA from a particular cell sample that combines template-switching PCR and T7-based amplification methods (referred to herein as CRL amplification protocol). While this method is preferably used for oocyte, cumulus, or ES total RNA samples it is applicable for any cell sample preferably a cell sample that is only available in small quantity.

The invention further provides transcriptome data obtained from oocyte, cumulus, or ES cells that identifies genes which are differentially expressed therein.

The invention in particular identifies 1626 genes that are differentially expressed by human ES cells.

The invention further identifies 5331 transcripts upregulated and 7074 transcripts down-regulated in human oocyte sample. Upregulated genes include FIGLA, STELLA, VASA, DAZL, GDF9, ZP1, ZP2, MOS, OCT4, NPM2, and H1FOO.

The invention further compares transcriptomes from human and mouse oocytes and identifies 1587 genes common (differentially expressed) to both.

The invention further compares the transcriptomes of oocytes and ES cella and identifies 388 (human) and 591 (mouse) genes differentially expressed in both as well as a set of 66 genes that are preferentially differentially expressed in each of human and mouse ESCs and oocytes.

In particular the invention provides a comprehensive expression baseline of gene transcripts present in in vivo matured metaphase II human oocytes.

In preferred embodiments, the inventive methods will be used to identify women subjects who produce or do not produce pregnancy competent oocytes based on the levels of expression of a set of differentially expressed genes. However, the inventive methods are applicable to non-human animals as well, e.g., other mammals, avians, amphibians, reptiles, et al. For example, the subject invention may be used to derive animal models for the study of putative female fertility treatments.

Additionally, the present invention may be used to identify female subjects who have an abnormality that precludes or inhibits their ability to produce pregnancy competent oocytes, e.g., ovarian dysfunction, ovarian cyst, pre-menopausal or menopausal condition, cancer, autoimmune disorder, hormonal dysfunction, cell proliferation disorder, or another health condition that inhibits or precludes the development of pregnancy competent oocytes.

For example, subjects who do not express specific pregnancy signature genes at characteristic expression levels will be screened to assess whether they have an underlying health condition that precludes them from producing pregnancy competent oocytes. Particularly, such subjects will be screened to assess whether they are exhibiting signs of menopause, whether they have a cancer, autoimmune disease or ovarian abnormality, e.g., ovarian cyst, or whether they have another health condition, e.g., hormonal disorder, allergic disorder, etc., that may preclude the development of “pregnancy competent” oocytes.

Additionally, the subject methods may be used to assess the efficacy of putative female fertility treatments in humans or non-human female subjects. Essentially, such methods will comprise treating a female subject, preferably a woman, with a putative fertility enhancing treatment, isolating at least one oocyte from said woman after treatment, optionally further isolating at least one oocyte prior to treatment, isolating at one cumulus cell from each of said isolated oocytes; detecting the levels of expression of at least one gene that is expressed or not expressed at characteristic levels by cumulus cells that are associated with (surround) pregnancy competent oocytes; and assessing the efficacy of said putative fertility treatment based on whether it results in cumulus cells that express at least one pregnancy signature gene at levels more characteristic of cumulus cells that surround pregnancy competent oocytes (than without treatment). As noted, while female human subjects are preferred, the subject methods may be used to assess the efficacy of putative fertility treatments in non-human female animals, e.g., female non-human primates or other suitable animal models for the evaluation of putative human fertility treatments.

Still further, the present invention may be used to enhance the efficacy of in vitro or in vivo fertility treatments. Particularly, oocytes that are found to be “pregnancy incompetent”, or are immature, may be cultured in one or more gene products that are encoded by “pregnancy signature” genes, e.g., hormones, growth factors, differentiation factors, and the like, prior to, during, or after in vivo, or in vitro fertilization. Essentially, these gene products should supplement for a deficiency in nutritional gene products that are ordinarily expressed by cumulus cells that surround “pregnancy competent” oocytes, and which normally nurture oocytes and thereby facilitate the capability of these oocytes to yield viable pregnancies upon fertilization.

Alternatively, one or more gene products encoded by said pregnancy signature genes may be administered to a subject who is discovered not to produce pregnancy competent oocytes according to the methods of the invention. Such administration may be parenteral, e.g., by intravenous, intramuscular, subcutaneous injection or by oral or transdermal administration. Alternatively, these gene products may be administered locally to a target site, e.g., a female ovarian or uterine environment. For example, a female subject may have her uterus or ovary implanted with a drug delivery device that provides for the sustained delivery of one or more gene products encoded by “pregnancy signature” genes.

Also, the novel CRL amplification protocol of the invention may be used to identify differentially expressed genes from any cell sample, preferably those only available in limited numbers such as e.g., samples used in forensic analysis, pathological samples such as cancer cells, especially cancer stem cells, cell samples suspected of containing an unknown pathogen, cell samples obtained from cells undergoing specific cellular processes such as differentiation, apoptosis, angiogenesis, and the like. This protocol has been found to faithfully and consistently amplify small amounts of RNA to quantities required for microarray analysis.

Thus, in general, the present invention involves the identification and characterization, in terms of gene identity and relative abundance, of genes that are expressed by desired cells, e.g., cumulus cells derived from an egg, preferably human egg, at the time of ovulation, preferably cumulus cells, the expression levels of which correlate to the capability of said egg to give rise to a viable pregnancy upon natural or artificial fertilization and transferal to a suitable uterine environment. Also, the invention identifies a set of genes differentially expressed by human or murine ESCs and metaphase II oocytes.

In one preferred embodiment, of the invention at least 50 to 100 genes that are significantly upregulated or downregulated, by cumulus cells that correlate to the “pregnancy competency”, of an oocyte from which said cumulus cells are associated with will be chosen and monitored in the inventive genetic testing methods.

However, while the invention preferably will select at least 50-100 genes from each of said categories, it is anticipated that the inventive methods alternatively may be practiced by monitoring the expression levels of fewer numbers of cumulus cell expressed genes, wherein said genes are similarly selected to be those which correlate to cumulus cells associated with “pregnancy competent” oocytes, i.e., those that are capable of yielding viable pregnancies.

According to the invention, gene expression levels will preferably be detected by the novel CRL amplification protocol provided herein. However other known methods, preferably real time detection methods such as mentioned above may be used to detect and quantify gene expression. Methods for detecting relative gene expression levels are known in the art and well within the purview of the ordinary skilled artisan.

As noted supra, this invention further provides a novel mRNA amplification protocol that is well suited for small cell samples such as a few or even a single cumulus cell or oocyte or ESC or other desired cell. This amplification protocol is well suited as well for forensic applications where only a minute nucleic acid sample may be available. Also, this technique is useful wherein only a few cells may be isolated from an individual such as adult stem cells, cancer stem cells, other differentiation specific cells, olfactory cells, taste cells, and the like. The present protocol will be useful in the biomedical field such as by medical and veterinary pathologists, e.g. in coordination with Laser-assisted Microdissection of tissues. Particularly, such applications may include cancer-related applications, research and disease diagnosis.

Previously, in order to generate a biotin-labeled antisense aRNA target for GeneChip experiments from limited amount of RNA samples, this entailed the use of commercial kits from several venders (such as Ambion TX, and Arcturus, CA) are available. All of these kits use the same approach based on the Eberwine T7 amplification method (See Eberwine Biotechniques 20:584-591 (1996)).

By contrast, the present invention provides an improvement thereover that faithfully and consistently amplifies small amounts of RNA to quantities required to perform microarray experiments. The CRL amplification protocol disclosed herein provides a practical approach to facilitate the analysis of gene expression in samples of small quantity while maintaining the relative gene expression profile throughout reactions (Kocabas et al., “Transcriptome Analysis of the Human Oocyte” In Press, 2006) which is incorporated by reference in its entirety herein.

The present amplification protocols achieve at least the following advantages:

(i) global mRNA amplification is possible for a limited number of cells, tissues and micro-dissected biopsy using other (non-CRL) PCR amplification method;

(ii) the protocol is comprised of simple laboratory manipulations;

(iii) the simplicity of the protocol contributes to a high level of reproducibility from experiment to experiment; and

(iv) the protocol time is shorter than other methods, in particular when multiple rounds are performed.

Based on these advantages this methodology is well suited for use in the present differential gene expression based-assays which detect genes the expression of which correlates to oocytes or embryonic stem cells as well as other applications wherein the detection of expressed genes in a sample is desired.

Essentially, the CRL protocol is depicted in FIG. 2 and comprises (i) first strand cDNA synthesis coupled with dC tailing by MMLV RT (Rnase H-); (ii) template switching and chain extension by RT; (iii) amplification of cDNA a requisite number of cycles, typically 5 to 50. more preferably around 10-20, more preferably around 15 cycles by LD-PCR using SMART primers; (iv) and production of double stranded DNAs by in vitro transcription using T7 RNA polymerase. As noted this methodology is applicable for amplification of mRNAs in any sample, but preferably is used in amplifying mRNAs from relatively small cell samples such as samples containing a few number of cumulus cells, oocytes, or stem cells.

In the inventive pregnancy signature gene detection methods, cumulus cells will be isolated from oocytes of different female subjects, the oocytes fertilized by known IVF procedures, and the cumulus cells of the corresponding isolated oocytes being subjected to gene expression analysis, i.e., by isolation of total RNA therefrom, amplification of said total RNA, quantification of the relative gene expression levels of said RNAs by microarray analysis and RT-PCR, and the identification of genes, the expression of which correlates to oocytes that give rise to a viable pregnancy.

To effect such identification, as a separate step, the status of embryos fertilized with oocytes derived from each of said cumulus cell samples will be monitored and pregnancy data recorded. Particularly, the relative birth rate and the health status of the newborn for each oocyte will be recorded and the gene expression levels of cumulus cells associated with each oocyte assessed as a function of pregnancy rate, newborn health, among other parameters, e.g., gender. Based on these results, a set of genes the expression of which correlates to pregnancy/health outcome or gender will be identified. (“pregnancy signature”)

This set of genes, and the corresponding expression levels is referred to herein as the “pregnancy signature” because these gene expression levels correlate to the development of a viable pregnancy and ultimately the production of a healthy newborn. While this “pregnancy signature” may comprise as many as 50, 100 or even 200 genes, it is anticipated that a fewer number of genes, e.g., on the order of 20 or less genes, may be sufficient to develop a suitable “pregnancy signature”.

The genes which constitute the “pregnancy signature” may include genes which encode gene products that are involved in the nutritional and developmental requirements of the oocyte, i.e., maturation and development, and the potential of the oocyte to be capable of yielding a viable pregnancy. These gene products may include growth factors, hormones, transcription factors, differentiation promoting agents, and the like. After the “pregnancy signature” is obtained, the corresponding genes are sequenced, the DNA sequences are then used to deduce the identify the corresponding polypeptide sequences, and these sequences then compared to databases of available human or other gene sequences to identify the identity of the gene products that correlate to the ability of an oocyte to yield a viable pregnancy. Genes which are differentially expressed by human oocytes are identified infra and include such pregnancy signature genes. Further statistical analysis of the relative levels of expression of these genes, or subsets of such genes, will identify preferred subsets of these genes that constitute a “pregnancy signature” of a viable oocyte, i.e., one that is pregnancy competent. The genes found to be differentially expressed in human cumulus cells are contained in SEQ ID NO's 1-513 infra.

As noted previously, these polypeptide gene products deficient in pregnancy incompetent oocytes may be added to in vitro culture media containing oocytes in order to enhance their pregnancy competency or alternatively may be administered in vivo as part of a fertility treatment regimen.

In order to further illustrate the advantages of the present invention, in particular those relating to the novel CRL amplification protocol the following example is provided. This exemplary is intended to be exemplary of the invention

### EXAMPLE 1

(Exemplification of CRL Amplification Protocol of the Invention with Oocyte and ES Cell Samples)

The mammalian oocyte is responsible for a number of extraordinary biological processes. It has the ability to haploidize its DNA, to reprogram sperm chromatin into a functional pronucleus, to drive early embryonic development and to give rise to pluripotent embryonic stem cells (ESCs). Identifying the genes in the oocyte essential for oogenesis, folliculogenesis, fertilization and early embryonic development will provide a valuable genomic resource in reproductive and developmental biology. However, the oocyte transcriptome and its functional significance in the human are relatively unknown due to ethical and technical limitations.

Attempts have been made to address this problem using candidate gene approaches employing reverse transcriptase-PCR (RT-PCR) and differential display 1-16. In addition, Serial Analysis of Gene Expression (SAGE) and complementary DNA (cDNA) libraries were generated from human oocytes, and SAGE tags and Expressed Sequence Tags (ESTs) were sequenced for rapid gene discovery and expression profiling in the oocytes (see reviews 17, 18). However these molecular approaches resulted in a small number of genes analyzed in each sample. DNA microarrays are relatively new technologies for whole-genome transcriptome analysis that when used in combination with reliable RNA amplification protocols, can be a powerful tool for the analysis of the oocyte transcriptome.

Extensive genomic studies of oocytes and preimplantation embryos have been conducted in mouse (19-24). However in human, the accessibility of mature oocytes i.e. metaphase II oocytes, is a major barrier to study oocyte genomics using microarrays. Recently, two reports described initial transcriptome analyses of human oocytes using microarrays with commercial RNA amplification kits (Arcturus, CA); a version of the Eberwine's T7 amplification method 25. The first study used discarded oocytes that failed to fertilize, and its array was limited to 8,793 transcripts 26. The second study employed mature human oocytes however its conclusions were based on only one reliable sample 27. Thus, the present study was conducted to identify the gene transcripts present in metaphase II oocytes within minutes after isolation from the ovary in three independent replicates and to compare these transcripts to a reference RNA (a mixture of total RNA from 10 different normal human tissues not including the ovary) using Affymetrix GeneChip technology. To achieve this goal, a novel protocol that combined template-switching PCR and T7 based amplification methods was developed for the analysis of gene expression in samples of small quantity. We amplified RNA from the oocyte and reference samples. Results were later compared with available transcriptome databases of mouse oocytes, and human and mouse ESCs.

Here, we report the transcript profile of in vivo matured human metaphase II oocytes using the most recent Affymetrix human GeneChip array interrogating over 47,000 transcripts including 38,500 well-characterized human genes.

Methods

Oocyte Collection, Total RNA Extraction and Reference RNA

Human oocytes were obtained from 3 patients undergoing an assisted reproductive treatment (ART) at the Unit of Reproductive Medicine of Clinica Las Condes, Santiago, Chile. The selection criteria for the donors was; a) less than 35 years old, b) reproductively healthy with regular ovulatory cycles, c) male factor as the only cause of infertility, and d) considerable number 0 developing follicles that assured spared oocytes. The experimental protocol was reviewed and approved by a local independent Ethics Review Board. All donors signed informed consent. At the time this manuscript was submitted, all three donors had already conceived; two of them got pregnant during the ART cycle in which our samples were collected, and the third one got pregnant following a spontaneous cycle with artificial insemination, using donated sperm. Ovarian stimulation and oocyte retrieval and isolation were performed as described in the supplemental information.

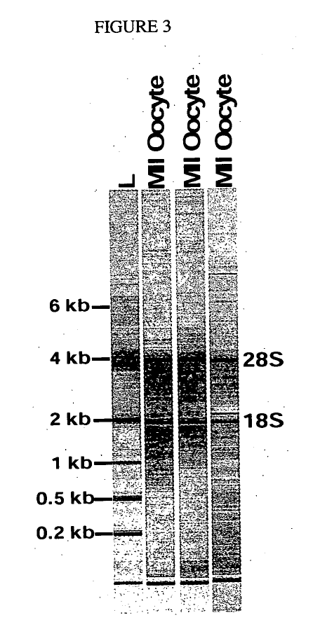

Three groups of 10 oocytes each were used. Total RNA was isolated following the guanidium thiocyanate method 28 using the PicoPure RNA isolation kit (Arcturus, CA) following manufacturer's instructions however only 6.5 •l elution buffer (Arcturus, CA) was used and the elution was repeated at least 3 times using the first eluate. All RNA samples within the purification column were treated with the RNase-Free DNase (Qiagen, CA). Extracted RNA was stored at −80° C. until used as template for cDNA synthesis. The quality and quantity of extracted total RNA from 8 matured oocytes (independent from the 30 oocytes used in this study) was evaluated on the Agilent 2100 bioanalyzer (Agilent Technologies, CA). Each mature oocyte was found to have about 330 pg total RNA when the Arcturus' RNA isolation kit was used. Quality of RNA was intact as shown in FIG. 3. Reference RNA (100 •g), was prepared by mixing 10 •g total RNA from each of 10 different normal human tissues including: skeletal muscle, kidney, lung, colon, liver, spleen, breast, brain, heart and stomach (Ambion, TX).

RNA Amplification for GeneChip Analysis (FIG. 3a)

First-strand cDNA synthesis: The following reagents were added to each 0.5 ml RNase-free tube: 5 •l total RNA (3 ng for the reference and 5 ®l, about 3 ng, for the oocyte samples) and 300 ng of an anchored T7-Oligo(dT)24 V promoter primer (Ambion, TX). The reaction tubes were incubated in preheated PCR machine at 70° C. for 2 min and transferred to ice. After denaturation, the following reagents were added to each reaction tube: 1.4 •l of SMART II A oligonucleotide (5′-AAGCAGTGGTATCAACGCAGAGTACGCGrGrGr-3′) (Clonetech, CA), 4 •l of 5× first-strand buffer, 2 •l of 20 mM DTT, 0.6 •l of 5 mg/ml T4 Gene 32 Protein (Roche, IN), 2•l of 10 mM dNTPs, 20 U RNase inhibitor (Ambion, TX) and 1 •l PowerScript Reverse Transcriptase (Clontech, CA). After gently mixing, reaction tubes were incubated at 42° C. for 60 min in a hot-lid thermal cycler. The reaction was terminated by heating at 70° C. for 15 min and purified by NucleoSpin Extraction Kit (Clonetech, CA).

Double-stranded cDNA synthesis by Long-distance (LD)-PCR, cDNA purification: PCR Advantage 2 mix (9 •l) was prepared as follows: 5 •l of 10×PCR Advantage buffer (Clontech, CA), 1 •l of 10 mM dNTPs, 100 ng 5′SMART upper primer (5′-AAGCAGTGGTATCAACGCAGAGTA-3′), 100 ng 3′SMART lower primer (5′-CGGTAATACGACTCACTATAGGGAGAA-3′), and 1 •l of Polymerase Mix Advantage 2 (Clontech, CA). This mix was added to 41 •l of the first-strand cDNA synthesis reaction product, and thermal cycling was carried out in the following conditions: 95° C. for 1 min, followed by 15 cycles, each consisting of denaturation at 94° C. for 30 sec, annealing at 62° C. for 30 see, and extension at 68° C. for 10 min. The cDNA was purified by NucleoSpin Extraction Kit following the manufacturer's instructions.

In vitro transcription (IVT), biotin labeled aRNA purification and aRNA fragmentation is described in supplemental information.

Microarray analysis: Transcriptional profile of each sample was probed using Affymetrix Human Genome U133 Plus 2.0 GeneChips. The raw data obtained after scanning the arrays was analyzed by dChip 29. A smoothing spline normalization method was applied prior to obtaining model-based gene expression indices, a.k.a. signal values. There were no outliers identified by dChip so all samples were carried on for subsequent analysis. More information on dChip is provided infra.

Pathways analysis was performed using Ingenuity Software Knowledge Base (Redwood City, Calif.), which is a manually created database of previously published findings on mammalian biology from the public literature. We used the network analysis using the knowledge base to identify interactions of input genes within the context of known biological pathways.

Gene Ontology (GO) was performed using EASE. Given a list of genes, EASE forms subgroups based on the functional categories assigned to each gene. EASE assigns a significance level (EASE score) to the functional category based on the probability of seeing the number of subgroup genes within a category given the frequency of genes from that category appearing on the microarray 30. (http://david.niaid.nih.gov/david/ease.htm)

Comparison with External Data Sets

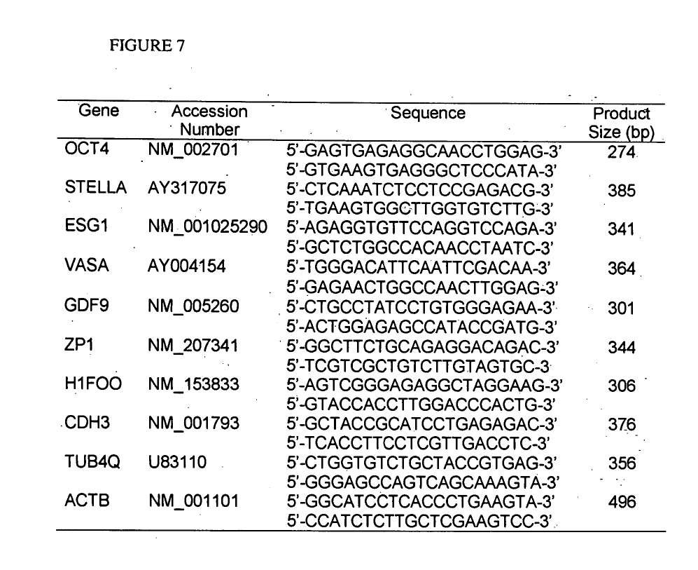

Mouse MII oocyte transcriptome data was obtained from Su et al. who used custom designed Affymetrix chips to obtain gene expression profiles of oocytes and 60 other mouse tissue types 31. Using their expression database, we identified 3,617 differentially upregulated transcripts in the mouse oocyte by using the median expression value of the remaining 60 samples as the baseline (FIG. 7). We selected transcripts with an expression value in oocyte samples that are two-fold higher than the baseline.

Human embryonic stem cell (hESC) data was derived from the work of Sato et al. who profiled human stem cells and their differentiated counterparts using Affymetrix HG-U133A representing ˜22,000 transcripts (32).

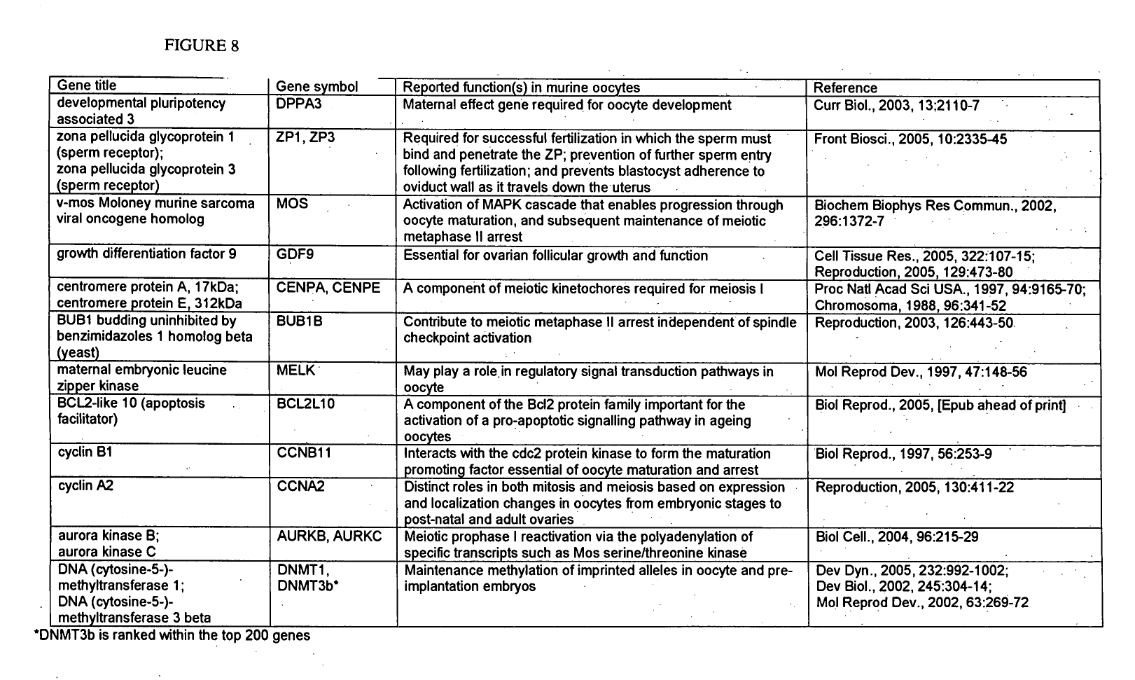

We analyzed the raw data using dChip and identified 1,626 hESC genes by selecting transcripts significantly up-regulated in human stem cells compared to their differentiated counterparts (FIG. 8).

Finally for mouse ES cells we used a list of 1,687 differentially upregulated mouse ES genes published by Fortunel et al (33) which were identified by comparing mouse ES cells to differentiated cells using Affymetrix MG-U74Av2 chips representing 12,000 transcripts (Supplementary dataset 3). We used Affymetrix NetAffx tool (https://www.affymetrix.com/analysis/netaffxlindex.affx) for mapping genes across organisms and platforms used in the respective studies. Supplementary datasets 1, 2, 3, 4, 5, 6, 7 and 8 can be downloaded at www.reprogramming.net.

Results

Validation of Amplification Fidelity (Amplified vs. Non-Amplified RNA)

A critical step in the analysis of gene expression on small samples is the faithful amplification of mRNA molecules present in the sample. We have designed a PCR based amplification system using the combination of SMART II A oligonucleotide (Clonetech, CA) and T7-Oligo(dT) promoter primers (CRL amplification protocol) (FIG. 2a). We isolated total RNA from a human cell line and 20, 3 and 1.5 ng input total RNA was amplified using CRL amplification protocol. For each experiment, 15 μg of fragmented aRNA was hybridized to a single Affymetrix Human Genome U133 Plus 2.0 array. Non-amplified RNA from the same original sample (1 μg) was run in parallel by using the MessageAmp II aRNA Kit (Ambion, TX). Gene expression results from both amplified vs. nonamplified RNA samples were compared and the correlation coefficients were found to be 0.94 (FIG. 2b), 0.93, and 0.91 for 20 ng, 3 ng, and 1.5 ng of total input RNA, respectively. CRL Amplification protocol was repeated two times with 20 ng initial total RNA from the same cell type and the correlation between the two experiments was 0.99. These results show that our RNA amplification strategy faithfully and consistently amplifies small amounts of RNA to quantities required to perform microarray experiments. The CRL amplification protocol provides a practical approach to facilitate the analysis of gene expression in samples of small quantity while maintaining the relative gene expression profile throughout reactions.

Differentially Upregulated Genes in the Human Oocyte

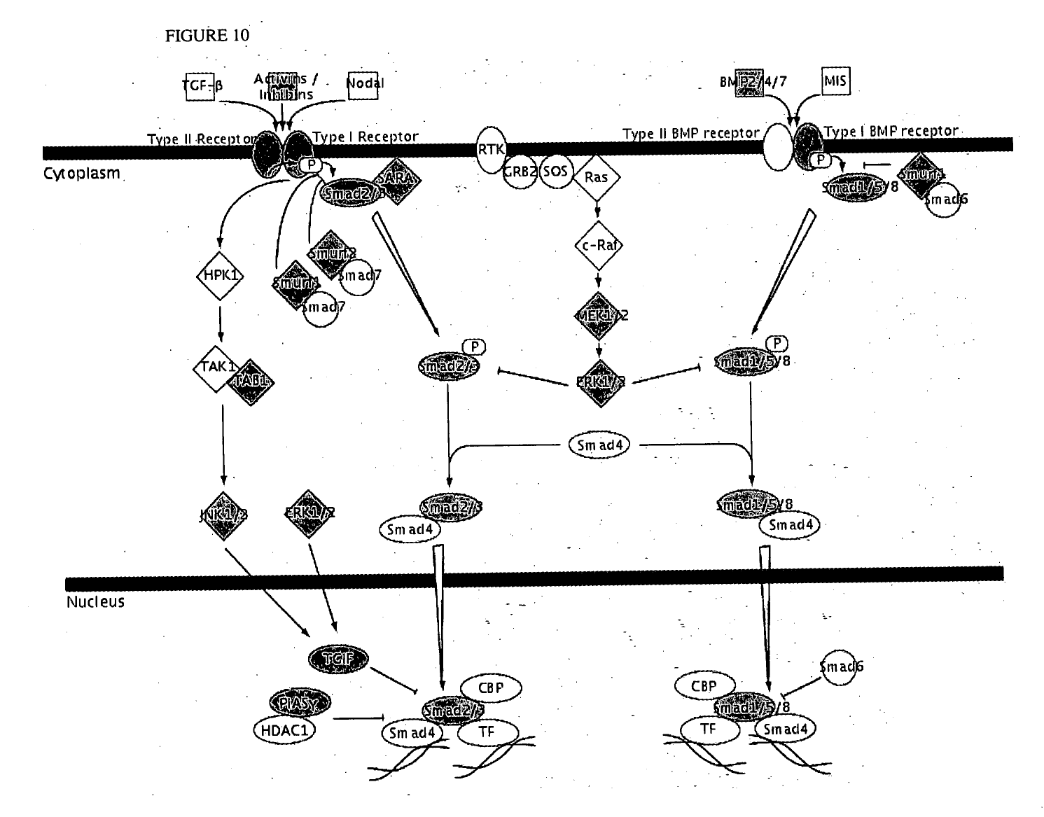

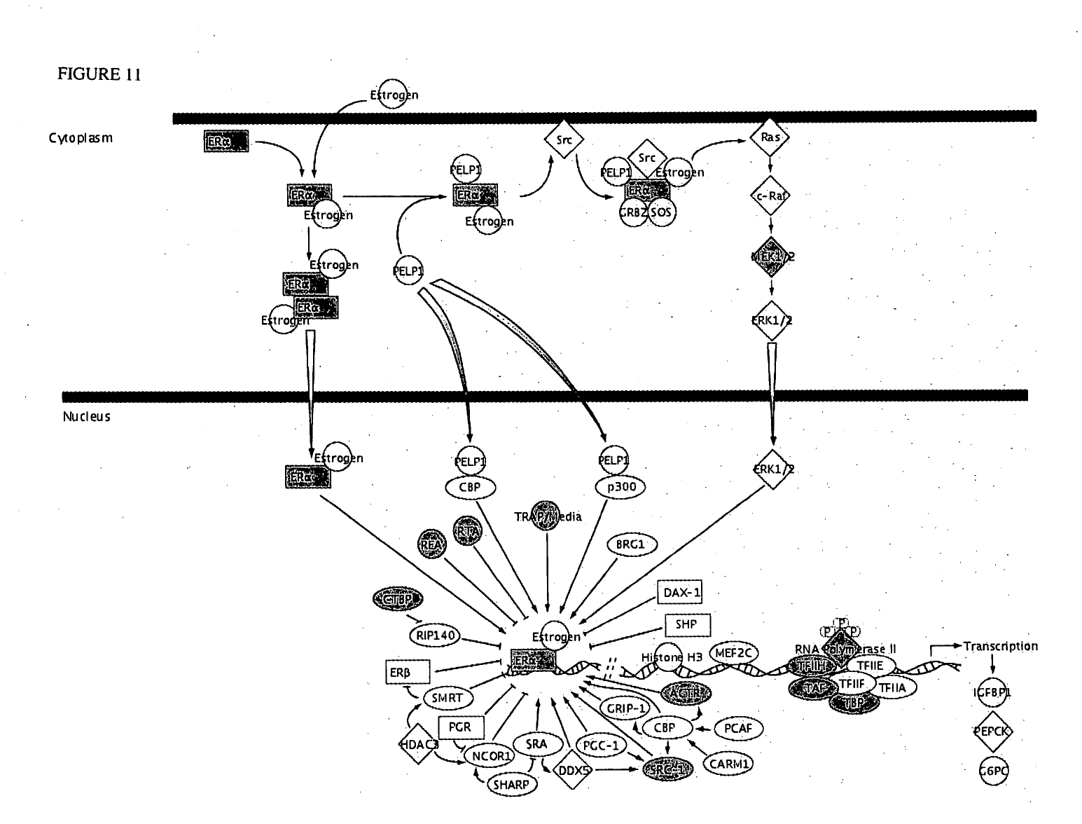

We generated a database of the human oocyte transcriptome by comparing the transcripts in the oocyte and the reference samples which contain mRNA from several somatic tissues. A complete list of up and down regulated genes, functional, comparative and correlation analysis is provided (see FIG. 10). Compared to reference samples there were 5,331 transcripts significantly up-regulated and 7,074 transcripts significantly down-regulated in the oocyte. Genes up-regulated in oocyte samples included most of the well-known germ cell specific genes, such as FIGLA, STELLA, VASA, DAZL, GDF9, ZP1, ZP2, MOS, OCT 4, NPM2, and H1FOO. Our analysis also confirms the presence of pathways previously described in the mouse, in particular the TGF-• pathway (FIG. 11).

Validation of Microarray Data

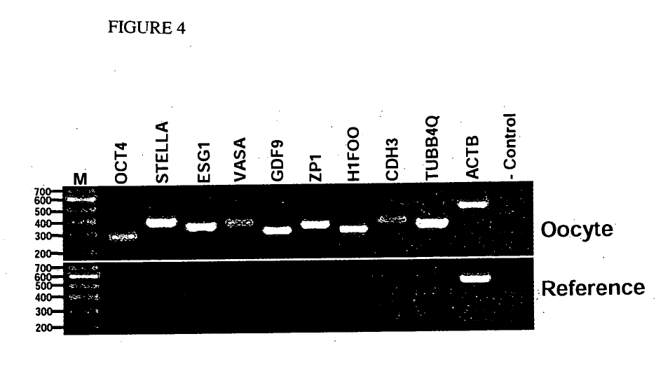

A selected list of genes known to be expressed in the oocyte was used to validate the microarray results by RT-PCR (FIG. 4). These genes were found to be present in the oocyte sample and absent in the reference RNA.

Functional annotation of genes over-expressed in the Human Oocyte

To examine the biological processes performed by the oocyte, we implemented EASE36, contrasting the genes over-expressed in the oocyte with all the genes present in Affymetrix chip (FIG. 7). One of the top over-represented categories found in oocytes was related to RNA metabolism. This is in agreement with the fact that oocytes store RNA to support the events of fertilization and early embryonic development until the embryonic genome is activated (34, 35 36). DNA metabolism and chromatin modification were also over-represented categories, in agreement with the need of the oocyte to remodel the sperm chromatin upon fertilization.

Cell cycle related categories were the most over-represented. Many genes known to be involved in the regulation of the meiotic cell cycle were detected (MOS, AKT2, CDC25, and PLK1) (37). Detection of gametogenesis and reproduction as over-represented categories further suggests the accuracy of this transcriptional profiling. Protein kinases and phophatases denoted another functional category over-represented in oocytes. Many of the cell cycle regulatory genes (AURKB, CDC25, CDC7, PLK1, CCND2, CDC23 and PLK3) and some receptors of the TGF beta superfamily (ACVR1, ACVR2B, and BMPR1A and 1B) were in this category.

An important category that is highly represented in the oocyte was related to nucleic acid metabolism and regulation of transcription. Although transcriptionally silent at the MII stage, the oocyte is very active in transcription and translation during its growth phase and must be prepared to initiate transcription at the time of embryonic genome activation, 4 to 8-cell stage in human 38. Many of the genes in this category represent Zinc-finger proteins that are not yet fully characterized, providing an opportunity to discover new transcriptional regulatory networks that operate during embryonic genome activation.

We also found that chromatin remodeling genes are well represented in the human oocyte. Genes in this category expressed in the human oocyte were: DNA methyltransferases (DNMT1, DNMT3A and DNMT3B), histone acetyltransferases (NCOA 1 and, 3, SRCAP, GCN5L2 and TADA2L), histone deacetylases (HDAC3, HDAC9 and SIRT7), methyl-CpG-binding proteins (MBD2 and MBD4), histone methyltransferases (EHMT1 and SET8), ATPdependent remodeling complexes (SMARCA 1, SMARCA5, SMARCAD1, SMARCC2 and SMARCD1) and other chromatin modifying genes (ESR1, NCOA6, HMGB3, HMGN1 and HMGA 1).

These GO results validate our transcriptome analysis when compared with candidate gene analysis already reported in other species but more importantly, shed new light into a large number of biological processes that take place in the human oocyte.

Intersection Between Human Oocyte and Mouse Oocyte Transcriptome

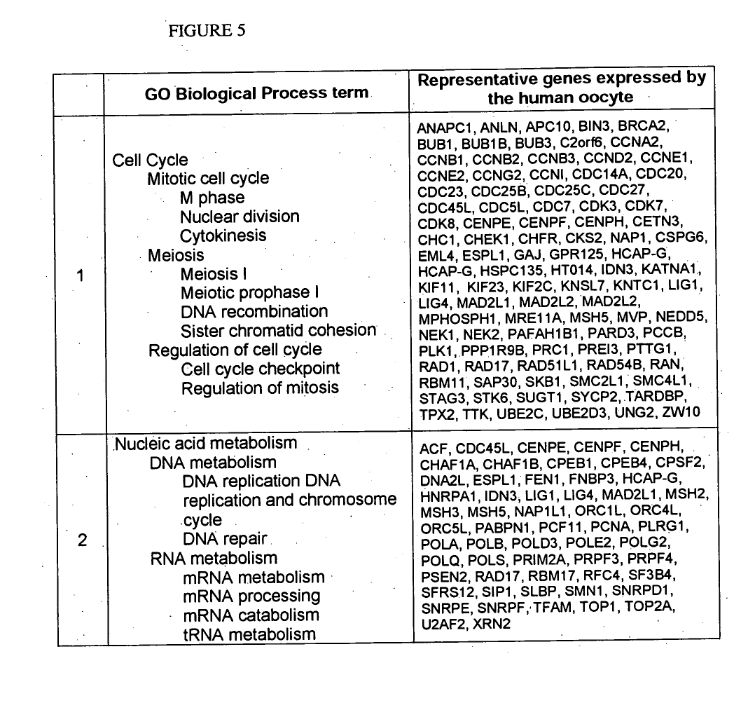

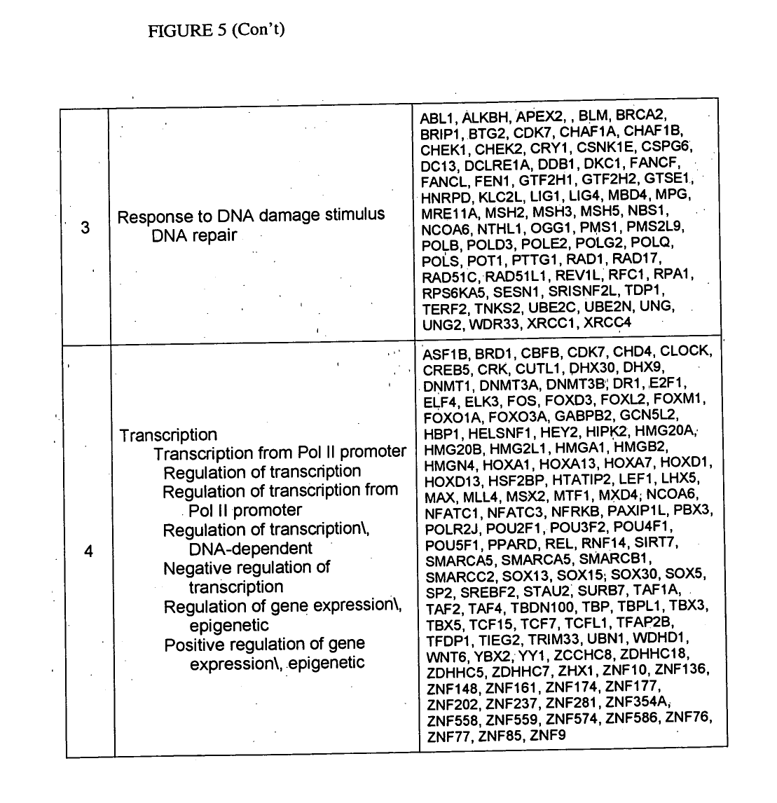

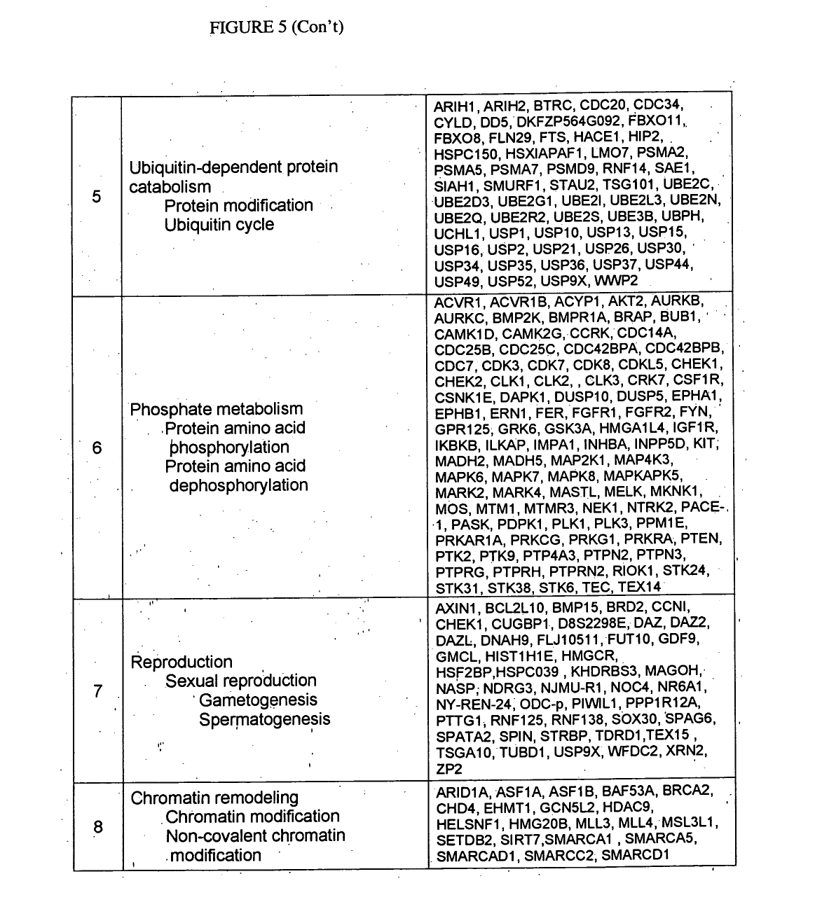

Mouse has been the best model for genetic studies and several groups have already reported the transcriptome analysis of mouse oocytes 39. In an effort to find differences and similarities between the human and mouse oocyte, we compared our human oocyte transcriptome results with that of mouse oocyte transcriptome derived from data of Su et al (35). The intersection of the two transcriptomes yielded a set of 1,587 genes to be common in both mouse and human oocytes, indicating genes of conserved function in mammalian oocytes (FIG. 5a). (FIG. 8 further shows the functional characterization of 16 of the top 100 intersected genes which have their functions described in mouse oocytes. Many of these genes relate to oocyte maturation, from the first meiotic division to Mil arrest, encompassing various controls of cell cycle checkpoints and cellular machinery for DNA segregation and cell division. Using the Ingenuity software to analyze the intersection of these two datasets we found that the estrogen receptor (ER) signaling pathway is represented in human and mouse oocytes (FIG. 11). Genes significantly upregulated in this pathway were CTBP2, ESR1, GTF2H1, GTF2H2, MAP2K1, NCOA1, NCOA3, PCQAP, PHB2, POLR2C POLR2J, RBM9, TAF3, TAF4 and 4B, TAF5, TAF6, TAF12 and TBP. Recent studies in KO models for aromatase have shown that estrogen is not required for the generation of preimplantation embryos 40 however our study suggests that in the oocyte some genes associated with the ER pathways are transcribed, perhaps in response to hormonal stimulation during folliculogenesis and oocyte maturation. Like with the EGF pathway, it remains to be determined whether the ER pathway has a role during preimplantation development in human embryos.

Considering the high degree of similarity in early embryonic development between mouse and human, these 1,587 common genes deserve particular attention, and must be considered for future candidate gene-approach studies related to fertility disorders, developmental defects and assisted reproductive technologies. Furthermore, with the inherent ethical and technical difficulties of studying human oogenesis in the laboratory, the mouse model will continue to provide a platform for the functional characterization of other highly conserved genes which may bear significance in understanding human germ cell formation and maturation.

Intersection Between Transcriptomes of the Oocytes and Embryonic Stem Cells

The oocyte is derived from germ cell-precursors believed to have segregated from pluripotent precursors prior to somatic tissues differentiation (41). Primordial germ cells (PGCs) undergo mitotic proliferation followed by meiosis. By the time the oocyte reaches the MII stage, it is already a highly specialized cell capable of remodeling the sperm nucleus and restoring totipotency to the diploid zygote. In addition, somatic cell nuclear transfer (SCNT) into enucleated oocytes have shown that when challenged with a somatic nucleus, the oocyte cytosol will attempt to completely erase the somatic epigenetic phenotype and transform the nucleus to a totipotent state. Although failures in this epigenetic reprogramming have been reported elsewhere, there are reported cases in which animals produced by SCNT have developed normally (42). Reinforcing the notion that when SCNT is performed under ideal circumstances (yet to be described), the oocyte cytosol can turn a somatic nucleus into a totipotent one.

Recent somatic cell-ESCs fusion experiments suggest that ESCs retain similar as yet undefined components that can initiate the reprogramming of introduced somatic nucleus to confer totipotency to the somatic nuclei (as measured by phenotypic and by transcriptional analyses). In this respect, the cytoplasmic environment of both ESCs and oocytes share the capacity to reprogram somatic nucleus (43-45). Furthermore, recent work suggests that mESCs can give rise to PGCs that can differentiate into cells similar to oocytes and sperm in a period of time significantly shorter when compared to in vivo gametogenesis (46, 47)

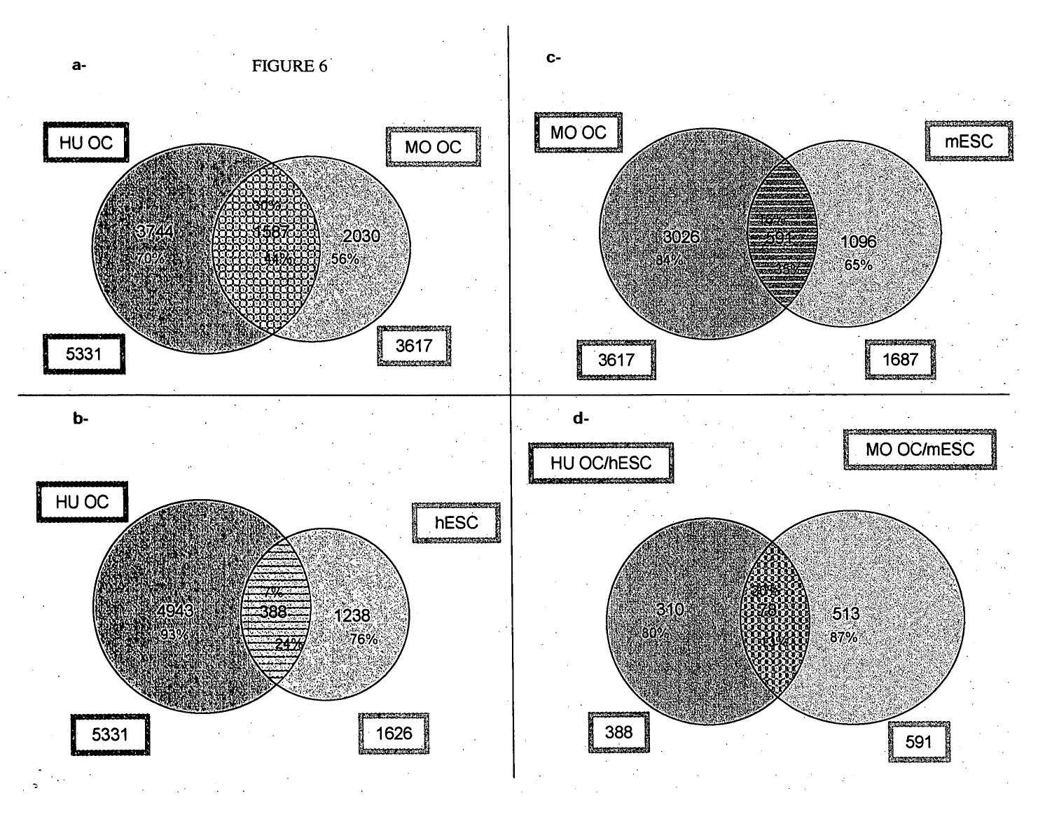

In order to identify genes that may reflect the similarities between ESCs and oocytes, differentially upregulated transcripts in the oocyte were compared and intersected with recently published data for genes that are expressed preferentially in ESCS 32. Our analysis of the Sato et al data revealed 1,626 hESCs differentially upregulated genes (see methods). When these hESCs genes were intersected with our human differentially upregulated oocyte transcripts, we found an overlap of 388 transcripts (FIG. 6b). In the mouse, a comparison of differentially upregulated oocyte and mESCs transcripts (see methods). A list of 591 genes common to mouse oocyte and mESCs was obtained (FIG. 6c).

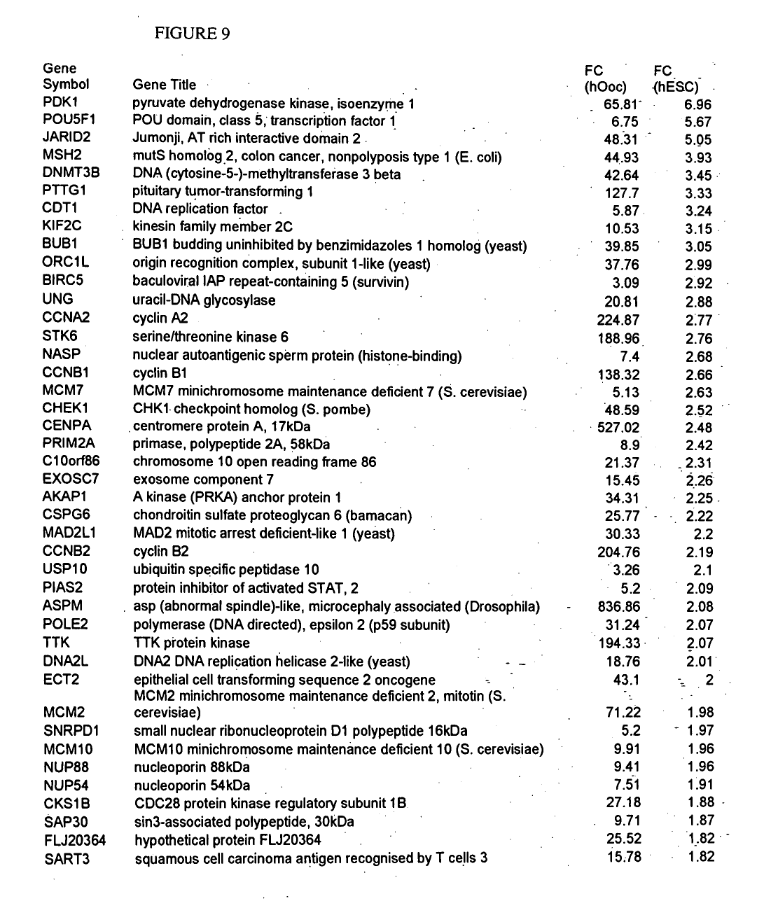

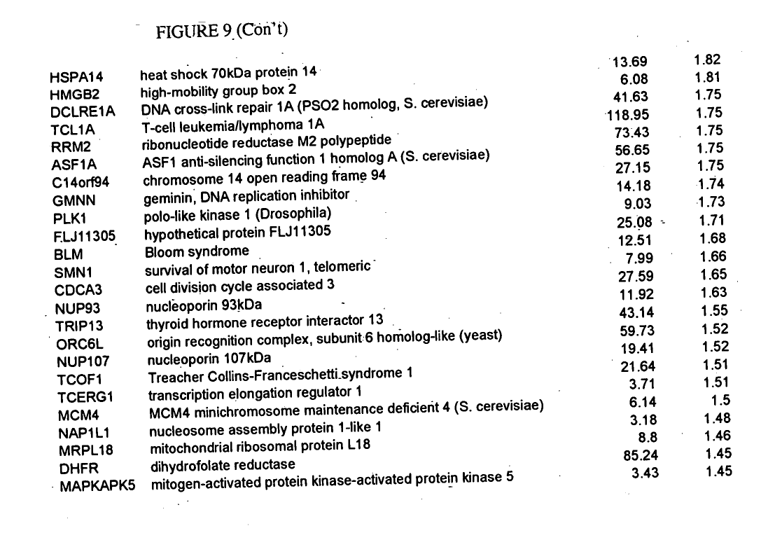

When compared with the human oocyte and hESC-shared transcripts, a list of 66 unique genes (78 transcripts) common amongst mouse oocyte, mESCs, human oocyte and hESCs was obtained. Five of these genes have unknown functions (FIG. 6d, FIG. 9). Among these 66 genes, there is a high abundance of chromatin and DNA modifying genes, suggesting that both the genome of the ESCs and oocytes are maintained in a responsive or primitive-native state, potentially primed for the activation of a whole repertoire of genes leading the generation of all tissue lineages. Whether any of these genes is involved in the ability to reprogram somatic nuclei would be interesting to explore.

### CONCLUSION

To our knowledge, this invention for the first time provides a comprehensive expression baseline of gene transcripts present in in vivo matured metaphase II human oocytes.

Using the most recent Affymetrix Human GeneChip we have identified 5,331 transcripts highly expressed in human oocytes, including well-know genes such as FIGLA, STELLA, VASA, DAZL, GDF9, ZP1, ZP2, MaS, OCT 4, NPM2, NALP5/MA TER, ZAR1 and H1 Faa. More importantly, 1,430 of these upregulated genes have unknown functions, arguing for the need for further studies aimed to elucidate the functional role of these genes in the human oocyte.

We have also identified a significant number of genes common between hESCs and MII oocytes. Such genes may provide the missing link between ESCs and MII oocytes and may serve as genetic resource to identify ESCs that have full potential for differentiation into an oocyte.

As in the case of many microarray studies, profiling of the genes in the tissues of interest is the first step of a comprehensive experimental approach towards dissecting biological processes and their players at the molecular level.

Further understanding of the biological role of these genes may expand our knowledge on meiotic cell cycle, fertilization, chromatin remodeling, gene regulation, lineage commitment, pluripotency, tissue regeneration, and morphogenesis. The practical implications of compiling gene expression information on human gametes and embryos would be enormous by bolstering efforts to solve problems from infertility to degenerative diseases.

Supplemental Information

Methods:

Ovarian stimulation was performed under a long protocol, Gn-RH analog suppression (Ieuprolide acetate, Lupron®-Abbott) in a daily subcutaneous (s/c) dose of 0.5 mg. Recombinant FSH (rFSH, Gonal-f®-Serono or Puregón®-Organon) was administrated in daily doses that ranged between 200-300 IU, starting the second day of the mense. Follicular growth and estradiol levels were monitored every two to three days until follicles had a mean diameter between 18 and 20 mm. Oocyte maturation was achieved by an injection of 10,000 IU of hCG (pregnyl® Organon). Oocytes were retrieved from the ovary by aspiration using guided transvaginal ultrasound thirty six hours after hCG administration. Three hours after retrieval, oocytes were denuded from surrounding corona and cumulus cells by a brief incubation (10-30 sec) in 80 IU/ml hyaluronidase solution (LifeGlobal, USA) and subsequent pipetting to completely eliminate other cells. Oocytes were then observed at high magnification to confirm maturity (metaphase II stage) and to confirm the absence of other cells. Each oocyte was rinsed in sterile PBS and lysed in 100 •l of extraction buffer (XB, Arcturus, CA) in a RNase/DNase/Pyrogen free 0.5 ml microcentrifuge tube. Each sample was incubated for 30 min at 42° C., centrifuged at 3,000 g for 2 min and stored in liquid nitrogen until use.

In vitro transcription (IVT), biotin labeled aRNA purification and aRNA fragmentation: The purified double-stranded cDNA containing the T7 promoter sequence was used as a template for IVT labeling assays in the presence of biotin labeled-ribonucleotides, using the BioArray HighYield RNA Transcript Labeling kit with T7 RNA polymerase (ENZO, NY) as described by the manufacturer. The biotin-labeled aRNA was purified using RNeasy mini columns (RNeasy Mini Kit, QIAGEN, CA). In vitro transcription of the cDNA for each replicate yielded 70-90 μg of biotinylated aRNA and 15 μg of the labeled aRNA was fragmented at 94° C. for 35 min in 1× fragmentation buffer (40 mM Trisacetate pH 8.1, 100 mM KOAc and 30 mM MgOAc).

Hybridization Washing, Staining and Imaging: The Affymetrix GeneChip system was used for hybridization, staining and imaging of the arrays. Hybridization cocktails of 300 μI containing 15 μg of fragmented biotin-labeled aRNA and biotinylated exogenous hybridization controls (50 pM control Oligo B2, Eukaryotic hybridization controls (BioB at 1.5 pM, BioC at 5 pM, BioD at 25 pM and CreX at 100 pM), herring sperm DNA (0.1 mg/ml), BSA (0.5 mg/ml) in buffer (100 mM MES, 1 M NaCI, 20 mM EDTA and 0.01% Tween-20) were hybridized to the GeneChip Human Genome U133 plus 2.0 array (Affymetrix, CA). Hybridizations were performed automatically and each array was pre-hybridized with all components except the fragmented biotin-labeled aRNA in a chamber at 45° C. for 15 min. with rotation at 60 rpm. The pre-hybridized array was then hybridized with the aRNA cocktail for 16 hrs under the pre-hybridization conditions. After hybridization, the cocktail was removed from chip and the array was filled with non-stringent wash buffer (6×SSPE and 0.01% Tween-20). The arrays were washed according to Affymetrix protocol on a Fluidics station using non-stringent and stringent (100 mM MES, 0.1 M NaCl and 0.01% Tween-20) wash buffers. For the detection of hybridized fragments, the array was stained using SAPE (streptavidin-linked to phycoerythrin) stain and antibody solutions. SAPE stain solution (600 μl) contained 2 mg/ml BSA, 10 μg/ml streptavidin Phycoerythrin (SAPE) in 100 mM MES, 1M NaCI and 0.05% Tween-20. The antibody solution (600 μl) also contained: 2 mg/ml BSA, 0.1 mg/ml goat IgG, μg/ml biotinylated anti-streptavidin antibodies in 100 mM MES, 1 M NaCI and 0.05% Tween-20. The order of staining is SAPE, antibody and second SAPE.

The arrays were scanned using Affymetrix's high density GeneArray Scanner 3000 and imaged using Affymetrix GeneChip Operating Software (GCOS). The GCOS expression data report was generated for each sample and used to judge the quality of sample preparation and hybridization. The report included information about noise, background and percentage of probe sets called present based on the manufacturer threshold and software settings. Information about performance of exogenously, added prokaryotic hybridization control genes such as BioB, BioC and BioD of the E. coli biotin synthesis pathway and the ratio of intensities of 3′ probes to 5′ probes for housekeeping genes such as GAPDH and •-actin were also included in the report.

dChip Analysis

When comparing two groups of samples to identify genes enriched in a given group, we used the lower confidence bound (LCB) of the fold change (FC) between the two groups as the cut-off criteria. If 90% LCB of FC between the two groups was above 2, the corresponding gene was considered to be differentially expressed (DE). LCB is a stringent estimate of the FC and has been shown to be the better ranking statistic 48. Recently, dChip's LCB method for assessing DE genes have been shown to be superior to other commonly used approaches, such as MAS 5.0 and Robust Multiarray Average (RMA) (49) based methods (50).

By using LCB, we can be 90% confident that the actual FC is some value above the reported LCB. It was suggested by a study exploring the accuracy and calibration of Affymetrix chips using custom arrays and quantitative reverse transcriptase real-time PCR assays that the chip analyses underestimate differences in gene expression (51). It is then assumed that those genes with an LCB above 2 most likely have an actual FC of at least 3 52.

RT-PCR: Equal amounts of the remaining LD-PCR reactions diluted 1:1 with sterile H20 were amplified by gene-specific primers (FIG. 5). After an initial incubation at 94° C. for 3 min, the RT-PCR was carried out at 94° C. for 45 sec, 60° C. for 30 sec, 72° C. for 40 sec for 30 cycles. Upon the completion of PCR, the reaction was incubated at 72° C. for an additional 10 min. RT-PCR products were electrophoresed on a 1.5% agarose gel and documented with a Gel Documentation System (Bio-Rad, CA).

### REFERENCES

The following additional references are cited supra in the examples and are incorporated by reference in their entirety herein.
1. Pitera J E. Milla P J, Scambler P. Adjaye J. Cloning of HOXDI from unfertilised human oocytes and expression analyses during murine oogenesis and embryogenesis. Mech Dev 2001; 109:377-81.
2. Verlinsky Y. Morozov G. Gindilis V, et al. Homeobox gene expression in human oocytes and preembryos. Mol Reprod Dev 1995; 41:127-32.
3. Adjaye J. Daniels R, Monk M. The construction of cDNA libraries from human single preimplantation embryos and their use in the study of gene expression during development. J Assist Reprod Genet 1998; 15:344-8.
4. Adjaye J. Bolton V, Monk M. Developmental expression of specific genes detected in high-quality cDNA libraries from single human preimplantation embryos. Gene 1999; 237:373-83.
5. Adjaye J. Monk M. Transcription of horn eo box-containing genes detected in cDNA libraries derived from human unfertilized oocytes and preimplantation embryos. Mol Hum Reprod 2000; 6:707-11.
6. Daniels R. Lowell S, Bolton V, Monk M. Transcription of tissue-specific genes in human preimplantation embryos. Hum Reprod 1997; 12:2251-6
7. Daniels R. Adjaye J, Bolton V, Monk M. Detection of a novel splice variant of the hypoxanthine-guanine phosphoribosyl transferase gene in human oocytes and preimplantation embryos: implications for a RT-PCR-based preimplantation diagnosis of Lesch-Nyhan syndrome. Mol Hum Reprod 1998; 4:785-9.
8. Hinkins M. Huntriss J, Miller D, Picton H M Expression of Poly comb-group genes in human ovarian follicles, oocytes and preimplantation embryos. Reproduction 2005; 130:883-8.
9. Liu H C. He Z Y, Tang Y X, et al. Simultaneous detection of multiple gene expression in mouse and human individual preimplantation embryos. Fertil Steril 1997; 67:733-41.
10. Salpekar A. Huntriss J, Bolton V, Monk M. The use of amplified cDNA to investigate the expression of seven imprinted genes in human oocytes and preimplantation embryos. Mol Hum Reprod 2001; 7:839-44.
11. Goto T. Adjaye J, Rodeck Cll, Monk M. Identification of genes expressed in human primordial germ cells at the time of entry of the female germ line into meiosis. Mol Hum Reprod 1999; 5:851-60.
12. Hsieh R H, Au H K, Yeh T S, Chang S J, Cheng Y F, Tzeng C R. Decreased expression of mitochondrial genes in human unfertilized oocytes and arrested embryos. Fertil Steril 2004; 81 Suppl 1:912-8.
13. Huntriss J. Hinkins M, Oliver B, et al. Expression of mRNAs for DNA methyltransferases and methyl-CpG-binding proteins in the human female germ line, preimplantation embryos, and embryonic stem cells. Mol Reprod Dev 2004; 67:323-36.
14. Huntriss J. Gosden R, Hinkins M, et al. Isolation, characterization and expression of the human Factor In the Germline alpha (FIGLA) gene in ovarian follicles and oocytes. Mol Hum Reprod 2002; 8: 1087-95.
15. Monk M. Holding C, Goto T. Isolation of novel developmental genes from human germ cell, oocyte and embryo cDNA by differential display. Reprod Fertil Dev 2001; 13:51-7.
16. Goto T. Jones G M, Lolatgis N, Pera M F, Trounson A O, Monk M. Identification and characterisation of known and novel transcripts expressed during the final stages of human oocyte maturation. Mol Reprod Dev 2002; 62:13-28.
17. Ko M S. Embryogenomics: developmental biology meets genomics. Trends Biotechnol 2001; 19:511-8.
18. Adjaye 1. Whole-genome approaches for large-scale gene identification and expression analysis in mammalian preimplantation embryos. Reprod Fertil Dev 2005; 17:37-45.
19. Brambrink T. Wabnitz P, Halter R et al. Application of cDNA arrays to monitor mRNA profiles in single preimplantation mouse embryos. Biotechniques 2002; 33:376-8, 380, 382-5.
20. Tanaka T S, Ko M S. A global view of gene expression in the preimplantation mouse embryo: morula versus blastocyst. Eur J Obstet Gynecol Reprod Biol 2004; 115 Suppl 1: S85-9l.
21. Wang Q T. Piotrowska K. Ciemerych M A, et al. A genome-wide study of gene activity reveals developmental signaling pathways in the preimplantation mouse embryo. Dev Cell 2004; 6:133-44.
22. Zeng F. Baldwin D A, Schultz R M. Transcript profiling during preimplantation mouse development. Dev Biol 2004; 272:483-96.
23. Pan H, O'Brien M J, Wigglesworth K, Eppig J J, Schultz R M. Transcript profiling during mouse oocyte development and the effect of gonadotropin priming and development in vitro. Dev Biol 2005; 286:493-506.
24. Hamatani T, Carter M G, Sharov A A, Ko M S. Dynamics of global gene expression changes during mouse preimplantation development Dev Cell 2004; 6: 117-31.
25. Eberwine J Amplification of mRNA populations using aRNA generated from immobilized oligo(dT)-T7 primed cDNA. Biotechniques 1996; 20:584-91.
26. Bermudez M G, Wells D, Malter H, Munne S, Cohen J, Steuerwald N M. Expression profiles of individual human oocytes using microarray technology Reprod Biomed Online 2004; 8:325-37.
27. Dobson A T, Raja R, Abeyta M J, et al. The unique transcriptome through day 3 of human preimplantation development. Hum Mol Genet 2004; 13:1461-70.
28. Chomczynski P, Sacchi N. Single-step method of RNA isolation by acid guanidinium thiocyanate-phenol-chloroform extraction. Anal Biochem 1987; 162:156-9.
29. Li C, Wong W H. Model-based analysis of oligonucleotide arrays: expression index computation and outlier detection. Proc Natl Acad Sci, USA 2001:98:316.
30. Hosack D A, Dennis G, Jr., Sherman B T, Lane H C, Lempicki R A. Identifying biological themes within lists of genes with EASE. Genome Biol 2003; 4:R70.
31. Su A I, Wiltshire T, Batalov S, et al. A gene atlas of the mouse and human protein encoding transcriptomes. Proc Natl Acad Sci, USA 2004; 101:6062-7.
32. Sato N, Sanjuan 1M, Heke M, Uchida M, Naef F, Brivanlou A H. Molecular signature of human embryonic stem cells and its comparison with the mouse. Developmental Biology 2003; 260:404-413.
33. Fortunel N O, Otu H H, Ng H H, et al. Comment on “‘Sternness’: transcriptional profiling of embryonic and adult stem cells” and “a stem cell molecular signature”. Science 2003; 302:393; author reply 393.
34. Bachvarova R. Gene expression during oogenesis and oocyte development in mammals. Dev Bioi (NY 1985) 1985; 1:453-524.
35. Wassarman P M, Kinloch R A Gene expression during oogenesis in mice. Mutat Res 1992; 296:3-15.
36. De La Fuente R, Eppig J J Transcriptional activity of the mouse oocyte genome: companion granulosa cells modulate transcription and chromatin remodeling. Dev Biol 2001; 229:224-36.
37. Kishimoto T. Cell-cycle control during meiotic maturation. Curr Opin Cell Biol 2003; 15:654-63.
38. Braude P, Bolton V, Moore S. Human gene expression first occurs between the four- and eight-cell stages of preimplantation development Nature 1988; 332:459-61.
39. Hamatani T, Carter M G, Sharov A A, Ko M S H. Dynamics of Global Gene Expression Changes during Mouse Preimplantation Development Developmental Cell 2004; 6:117-131.
40. Huynh K, Jones G, Thomas G, Britt K L, Simpson E R, Jones M E. Estrogen is not directly required for oocyte developmental competence. Bioi Reprod 2004; 70:1263-9.
41. Cauffman G, Van de Velde H, Liebaers I, Van Steirteghem A DAZL expression in human oocytes, preimplantation embryos and embryonic stem cells. Mol Hum Reprod 2005; 11:405-11.
42. Lanza R P, Cibelli J B, Faber D, et al. Cloned cattle can be healthy and normal. Science 2001; 294:1893-4.
43. Byrne J A, Simonsson S, Western P S, Gurdon J B. Nuclei of adult mammalian somatic cells are directly reprogrammed to oct-4 stem cell gene expression by amphibian oocytes. Curr Biol 2003; 13:1206-1213.
44. Cowan C A, Atienza J, Melton D A, Eggan K. Nuclear reprogramming of somatic cells after fusion with human embryonic stem cells. Science 2005; 309:1369-73.
45. Tada M, Takahama Y, Abe K, Nakatsuji N, Tada T. Nuclear reprogramming of somatic cells by in vitro hybridization with ES cells. Curr Bioi 2001; 11:1553-8.
46. Hubner K, Fuhrmann G, Christenson L K, et al. Derivation of Oocytes from Mouse Embryonic Stem Cells. Science 2003:1083452.
47. Geijsen N, Horoschak M, Kim K, Gribnau J, Eggan K, Daley G Q. Derivation of embryonic germ cells and male ganletes from embryonic stem cells. Nature 2003/2004; AOP: 1-6.
48 Li C, Hung Wong W. Model-based analysis of oligonucleotide arrays: model validation, design issues and standard error application. Genome Biol 2001; 2: RESEARCH0032.
49. Irizarry R A, Bolstad B M, Collin F, Cope L M, Hobbs. B, Speed T P. Summaries of Affymetrix GeneChip probe level data. Nucleic Acids Res 2003; 31:e15.
50. Shedden K, Chen W, Kuick R, et al. Comparison of seven methods for producing Affymetrix expression scores based on False Discovery Rates in disease profiling data. BMC Bioinformatics 2005; 6:26.
51. Yuen T, Wurmbach E, Pfeffer R L, Ebersole B J, Sealfon Sc. Accuracy and calibration of commercial oligonucleotide and custom cDNA microarrays. Nucleic Acids Res 2002; 30:e48.
52. Ramalho-Santos M, Yoon S, Matsuzaki Y, Mulligan R C, Melton D A. “Sternness”: transcriptional profiling of embryonic and adult stem cells. Science 2002; 298:597-600.

The following Figures are cited in the example above.

FIG. 3. Digital RNA gel-like image. MII Oocyte; size distribution of the total RNA sample isolated from 8 matured human oocytes run 3 times. The 28S and 18S ribosomal RNA bands are clearly visible in the intact RNA samples from the mature oocytes. L indicates RNA 6000 ladder.

FIG. 2.a—Flow Chart of CRL amplification protocol; 2b. Representative plot of gene intensities comparing CRL and Ambion amplification methods.

FIG. 4. RT-PCR verification of the GeneChip array result. Loading orders of the gel were as following: M, 100 bp molecular weight standards with sizes as indicated on the left margin; OCT 4, POU domain, class 5, transcription factor 1; STELLA, DPPA3, developmental pluripotency associated 3; ESG1, embryonal stem cell specific gene 1; VASA, DEAD box RNA helicase; GDF9, growth differentiation factor 9; ZP1, zona pellucida glycoprotein 1; H1 FOO, H1 histone family, member 0, oocyte-specific; CDH3, cadherin 3, type 1, P-cadherin (placental); TUB4Q, beta-tubulin; ACTB, beta actin; and negative control with no DNA template.

FIG. 5: Selected overrepresented GO biological processes in oocytes identified by EASE (EASE score <0.05).

FIG. 6. Venn diagrams depicting; a—intersection between DE genes in the human (HU OG) and mouse oocyte (MO OG). 1,587 transcripts Were found to be in common in both species; b—intersection between DE genes in HU OC and hESCs, 388 transcripts were found to be common in both cell-types; c—intersection between DE genes in MO OC and mESCs, 591 transcripts were found to be common in both cell-types; d—intersection between DE genes in HU OC/hESC and MO OC/mESC, 78 transcripts were found to be common in all 4 cell-types.

FIG. 7 Information of the primers and their sequences used for the validation of the microarray experiment by RT-peR.

FIG. 8: Selected representations of genes common between human oocyte and mouse oocyte with homologues functionally characterized in the mouse oocyte (16 selected genes out of the top 100).

FIG. 9. List of 66 unique genes in common among human oocytes, mouse oocytes, hESCs and mESCs.

FIG. 10. TGF-beta Signaling Pathway. Genes shown in red are differentially upregulated in the human oocytes.

FIG. 11. Estrogen Receptor Signaling Pathway. Genes shown in red are differentially upregulated in the human and mouse oocytes.

### EXAMPLE 2

Description

Phase I: At the clinic, embryologists will remove the cumulus cells of two eggs and fertilize them. Embryos will be transferred to the uterus of a woman and cumulus cells sent to the laboratory for analysis. Once the cells arrive to the laboratory, RNA will be isolated and microarray analysis performed using Affymetrix platform. Pregnancy tests will be done by ultrasound on day 30 and embryonic sacs counted. There will be three kinds of outcomes: 1) 0 sacs; 2) 1 sac and 3) 2 sacs. A minimum of 30 volunteer women will participate during this phase. Ten with no sacs, ten with one sac and ten with 2 sacs. Pregnancy data will be correlated with gene expression obtained from the cumulus cells isolated from those same eggs. One hundred genes that directly correlate with pregnancy—either by upregulation or downregulation—will be further analyzed using real time RT-PCR. The best 20 genes that correlate with pregnancy (positively or negatively) will be called “pregnancy signature” and used for later testing at the clinic.

Phase II: Blind validation of genes in the pregnancy signature. At the clinic, the embryologist will isolate RNA from cumulus cells from each oocyte that will be later fertilized. Half of the RNA will be sent to our laboratory and the rest will be used for real time RT-PCR analysis to be performed on site. Gene expression of the “pregnancy signature” will be measured. Embryologists will transfer embryos without knowing the outcome of gene expression analysis. One hundred women will be asked to participate as volunteers in this part of the study. At the time pregnancy results are obtained, the study will be unmasked and results from each individual will be correlated with gene expression analysis. We anticipate that the “pregnancy signature” put forward in phase 1 will be validated during this phase.

Alternative strategy: In the event of an unexpected outcome i.e., the pregnancy signature is not validated; microarray analysis will be run once more using the RNA provided by the clinic in phase 2. It is anticipated that having 100 more samples will result in the identification of a clear pattern of gene expression in cumulus cells from eggs capable (or non-capable) of generating a healthy pregnancy/baby.

Using microarray analysis as described above, the genes identified infra were found to be differentially expressed by cumulus cells obtained from eggs of women donors. The expression of those particular genes which correlate to pregnancy (positive or negative) will establish a “pregnancy signature”, i.e., genes the expression or absence of expression of which correlates to a positive pregnancy outcome and “infertility signature”, i.e., specific genes the expression or absence of expression correlate to fertility problems or abnormalities.

This is effected preferably by microarray analysis. For example, comparison of expression between two samples on filter arrays may be performed by comparing nucleic acids obtained from normal oocyte cells to those obtained from a donor suspected of having ovarian dysfunction that renders oocytes pregnancy incompetent on two duplicate filters or alternatively a single filter may be used that is stripped and hybridized sequentially.

Direct comparison of gene expression in two samples can be achieved on glass arrays by labeling the two samples with different flourophores. This technique allows the evolution of repression of gene expression as well as induction of expression. The two flouresently-labeled cDNAs are then mixed and hybridized on a single glass or filter array. Glass arrays have the advantage of allowing the simultaneous analysis of two samples on the same array under the same hybridization conditions.

Gene arrays containing sequences of genes implicated in pregnancy (“pregnancy signature”) will allow high-throughput screening of individuals for diagnostic purposes or tailor-made treatments.

Arrays of polynucleotide, the expression of which corresponds to, or are complementary to the sequences of genes identified by the method of the invention therefore provide a further aspect of the invention. Such an array will include at least two nucleic acid sequences, preferably at least 10, and more preferably at least 20, e.g., 50 genes or more that correspond to the sequence of, or are complementary to genes, the expression of which (positive or negative) the positive pregnancy outcome in cells obtained from oocyte donors, e.g., women suspected to have ovarian dysfunction as a result of disease, age, and the like. Protein arrays form a further aspect of the invention and will contain polypeptides encoded by such pregnancy signature genes or antibodies which bind thereto.

Recent developments in the field of protein and antibody arrays allow the simultaneous detection of a large number of proteins. As noted previously using these methods the following genes were found to be differentially expressed in the cumulus cell samples assayed as described above. The expression of these cumulus cell genes or a subset thereof will be used to identify the “pregnancy signature” i.e., the genes the level of expression of which correlates to the ability of an oocyte from a host from which the cumulus cells are derived to initiate and sustain a viable pregnancy. Set forth below are 513 genes which were found to be differentially expressed in the huma cumulus cells assayed as described above. These genes or a subset thereof, e.g., on the order of 25-50 genes, more preferably 5-15 genes, may be used to identify a putative pregnancy signature, i.e., the genes for which differential expression correlates to the ability of oocytes associated therewith to be capable of giving rise to a normal pregnancy, (pregnancy competent).

LENGTHY TABLE REFERENCED HERE
US20070054289A1-20070308-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070054289A1). An electronic copy of the table will also be available from the USPTO upon request and payment of the fee set forth in 37 CFR 1.19(b)(3).

1. A method of identifying oocytes that are capable of giving rise to a viable pregnancy when fertilized comprising the following steps:
(i) obtaining at least one cell associated with an oocyte;
(ii) assaying the expression of at least one gene by said cell, the expression of which correlates to the capability of an oocyte associated with said cell to yield a viable pregnancy upon fertilization and transferal into a suitable uterine environment; and
(iii) identifying, based on the level of expression of said at least one gene, whether said oocyte is potentially capable of yielding a viable pregnancy upon fertilization and transferal into a suitable uterine environment.

2. The method of claim 1, wherein said oocyte is a mammalian oocyte.

3. The method of claim 2, wherein said oocyte is a human oocyte.

4. The method of claim 2, wherein said oocyte is a non-human primate oocyte.

5. The method of claim 1 wherein said oocyte associated cell is a cumulus cell.

6. The method of claim 1, wherein the expression of at least 5 genes, the expression of which correlates to the capability of an oocyte to potentially yield a viable pregnancy are identified.

7. The method of claim 6, wherein the expression of at least 10 genes, the expression of which correlates to the capability of an oocyte to potentially yield a viable pregnancy are measured.

8. The method of claim 7, wherein the expression of at least 15 genes, the expression of which correlates to the capability of an oocyte to potentially yield a viable pregnancy are identified.

9. The method of claim 8, wherein the expression of at least 20 genes, the expression of which correlates to the capability of an oocyte to potentially yield a viable pregnancy are identified.

10. The method of claim 9, wherein the expression of at least 20 to 50 genes, the expression of which correlates to the capability of an oocyte to potentially yield a viable pregnancy are identified.

11. The method of claim 10, wherein the expression of at least 50 to 100 genes, the expression of which correlates to the capability of an oocyte to potentially yield a viable pregnancy are identified.

12. The method of claim 1 wherein the method of assaying gene expression uses a method that monitors differential gene expression.

13. The method of claim 12 wherein said method comprises indexing differential display reverse transcriptase polymerase chain reaction (DDRT-PCR)

14. The method of claim 3, wherein the oocyte is obtained from a woman who is at least 25 years old.

15. The method of claim 14, wherein the oocyte is obtained from a woman who is at least 30 years old.

16. The method of claim 15, wherein the oocyte is obtained from a woman who is at least 35 years old.

17. The method of claim 16, wherein the oocyte is obtained from a woman who is at least 40 years old.

18. The method of claim 3, wherein the aberrant expression of said at least one gene is correlated to a condition selected from menopause, cancer, ovarian dysfunction, ovarian cyst, autoimmune disorder and hormonal dysfunction.

19. A method of assessing the efficacy of a fertility treatment comprising:
(i) treating a woman with a putative fertility enhancing treatment;
(ii) obtaining an oocyte from said woman after treatment and measuring the expression of at least one gene by a cell obtained from said oocyte, the expression of which positively or negatively correlates to the capability of an oocyte cell to yield a viable pregnancy upon fertilization and transferal to a suitable uterine environment; and
(iii) evaluating whether said treatment is effective based on the level of expression of said at least one gene.

20. The method of claim 19, wherein said fertility treatment comprises hormonal therapy.

21. The method of claim 20, wherein the subject is menopausal and the treatment comprises hormone replacement therapy.

22. The method of claim 1, wherein gene expression is detected by real-time polymerase chain reaction (RT-PCR).

23. The method of claim 19, wherein gene expression is detected by RT-PCR.

24. The method of claim 19 wherein gene expression is detected differentially by indexing differential display reverse transcriptase polymerase chain reaction (DDRT-PCR).

25. The method of claim 19 wherein said cell is a cumulus cell.

26. A method of evaluating fertility potential in a subject comprising comparing the expression levels of specific genes in a cell obtained from an oocyte, wherein said genes are expressed at characteristic levels (upregulated or downregulated) in an oocyte capable of yielding a viable pregnancy; and determining whether said subject is potentially “pregnancy competent” based on whether a cell derived from an oocyte of said subject expresses one or more pregnancy signature genes at levels characteristic of pregnancy competent oocytes.

27. The method of claim 26 wherein said cell is a cumulus cell.

28. The method of claim 26 wherein said method monitors differential gene expression by indexing differential display reverse transcriptase polymerase chain reaction (DDRT-PCR).

29. The method of claim 1 wherein gene expression is detected using antibodies that specifically bind to “pregnancy signature” polypeptides.

30. An array of at least two different polynucleotide sequences or polypeptides encoded thereby the levels of expression or absence of expression correlate to the pregnancy potential of a mammalian oocyte expressing said polynucleotide sequences or polypeptides.

31. The array of claim 30 which is a polynucleotide sequence array.

32. The array of claim 30 which is a polypeptide array.

33. The array of claim 31 which comprises at least 10 polynucleotide sequences.

34. The array of claim 32 which comprises at least 10 polypeptides.

35. A method of using an array according to claim 30 to detect whether a cell expresses pregnancy signature genes.

36. A method of using an array according to claim 30 to determine the efficiency of a fertility treatment by detecting whether cells obtained from a fertility treatment subject express one or more nucleic acid or polypeptides contained in said array.

37. The array of claim 30 which is comprised on a filter or glass chip.

38. The array of claim 37 which comprised a detectable label.

39. The array of claim 38 wherein said label is a fluorophore.

40. An improved method for identifying the genes which are differentially expressed by a particular cell sample (whole genome transcriptome analysis) which method includes the amplification of total RNA from a cell sample wherein the improvement comprises:
effecting RNA amplification by a protocol which combines template-switching polymerase chain reaction (PCR) and T7-based amplification methods.

41. The method of claim 40 wherein first strand cDNA synthesis is effected by contacting total RNA from a cell sample with a T7 promoter primer, denaturing, and the addition of a SMART II oligonucleotide.

42. The method of claim 41 wherein the T7 promoter primer is an anchored T7-oligo (dT)₂₄ V promoter primer.

43. The method of claim 41 wherein the SMART II oligonucleotide is 5′-AAGCAGTGGTATCAACGCAGAGTACGCGrGr-3′ (SEQ ID NO: 517).

44. The method of claim 41 wherein after first strand cDNA synthesis is completed double stranded cDNA synthesis is effected by long-distance (LD-PCR).

45. The method of claim 44 wherein LD-PCR is effected by contacting the product of first strand cDNA synthesis with a 5′ SMART upper primer and a 3′ SMART lower primer and effecting a number of thermal cycles wherein each cycle each includes denaturation, annealing and extension steps.

46. The method of claim 45 wherein the number of thermal cycles ranges from 5 to 50.

47. The method of claim 46 wherein the number of thermal cycles ranges from 10-30.

48. The method of claim 8 wherein about 15 thermal cycles are effected.

49. The method of claim 47 wherein after second strand synthesis is effected by LD-PCR the resultant transcription product obtained from the cell sample is probed in order to identify the expressed genes contained therein.

50. The method of claim 49 wherein said probing is effected using a gene chip containing a set of genes specific to the cell sample.

51. The method of claim 50 wherein the gene chip comprises genes from the human genome or a defined subset thereof.

52. The method of claim 51 wherein the expression data is analyzed by dChip, MAS 5.0 or Robust MultiArray Average (RMA) based methods.

53. The method of claim 52 wherein the raw data is normalized prior to obtaining model-based gene expression indices or signals for the cell sample.

54. The method of claim 52 wherein the genes which are expressed greater than the baseline average are identified.

55. The method of claim 54 wherein the genes which are expressed at least 1.5-5 times greater than baseline are identified.

56. The method of claim 55 wherein the genes expressed at least 2 times baseline are identified.

57. The method of claim 40 wherein the cell sample is that of an oocyte, embryonic stem cell (ESC), primordial germ cell, sperm cell, oocyte or cumulus cell.

58. The method of claim 40 wherein the cell sample is from an oocyte, cumulus cell, or ESC.

59. The method of claim 58 wherein the cell sample is from a human oocyte, ES cell, or cumulus cell.

60. The method of claim 59 wherein the cell sample is from a human metaphase II oocyte.

61. The method of claim 60 wherein the set of expressed genes is used to identify a set of genes which are or are not expressed in pregnancy competent oocytes.

62. The method of claim 61 wherein the genes are selected from those contained in FIG. 4.

63. The method of claim 61 wherein the genes are selected from those contained in FIG. 5.
